(19) United States
(12) Patent Application Publication
(10) Pub. No.: US 2026/0311890 A1
(43) Pub. Date: Oct. 8, 2026

(54) INFORMATION GENERATION DEVICE, INFORMATION PROVISION SYSTEM, INFORMATION GENERATION METHOD, AND RECORDING MEDIUM

(71) Applicant: NEC CORPORATION, Tokyo (JP)

(72) Inventors: Chenhui HUANG, Tokyo (JP); Fumlyuki NIHEY, Tokyo (JP); Hiroshi KAJITANI, Tokyo (JP); Makoto YASUKAWA, Tokyo (JP); Yoshitaka NOZAKI, Tokyo (JP); Kosuke NISHIHARA, Tokyo (JP); Kenichiro FUKUSHI, Tokyo (JP); Kentaro NAKAHARA, Tokyo (JP); Azusa FURUKAWA, Tokyo (JP); Hiroaki NAKANO, Tokyo (JP); Kazuya OZAKI, Tokyo (JP)

(73) Assignee: NEC CORPORATION, Tokyo (JP)

(21) Appl. No.: 19/479,494

(22) PCT Filed: Jun. 23, 2023
(86) PCT No.: PCT/JP2023/023268

§ 371 Date: Oct. 28, 2025

(51) Int. Cl. G16H 50/80

(52) U.S. Cl. CPC G16H 50/80

(57) ABSTRACT

An information generation device comprising an acquisition unit that acquires sensor data measured by a measurement device mounted on footwear of at least one subject, a risk estimation unit that estimates the disease risk for each disease relating to at least one subject using the acquired sensor data, a map generation unit that generates a risk map in which a display corresponding to the disease risk for a target disease relating to at least one subject is overlaid on a map of a target district, and an output unit that outputs risk information including the generated risk map. The risk map provides information to support decision-making on city planning measures for the target districts.

### TECHNICAL FIELD

The present disclosure relates to an information generation device, an information provision system, an information generation method, and a recording medium.

### BACKGROUND ART

In areas distant from cities, the public burden of local governments is increasing due to an increase in the number of elderly people. In such areas, traffic is often inconvenient. Improvement of convenience of activities such as shopping related to the life of the elderly person, visiting a hospital, nursing care, and home visit medical care is determined by city planning measures. If the city planning that improves the convenience of these activities can be achieved, the health condition of the local residents and the attractiveness of the area can be expected to be improved. However, in reality, it is difficult to take such measures since it is difficult to grasp the life and health condition of the local residents.

PTL 1 discloses a health information management server that provides an analysis result of health information related to health for each region narrower than the entire analysis target area. The server of PTL 1 acquires health information on health of a resident from a resident terminal. The server of PTL 1 stores the acquired health information in the storage unit in association with the resident related to the health information. The server of PTL 1 analyzes health information relevant to each resident and calculates an individual health risk of the resident. The server of PTL 1 calculates an area health risk for each of a plurality of preset areas based on the calculated individual health risk and the resident's residential location. PTL 1 discloses that a degree of an area health risk calculated for each area is superimposed on a relevant area on a map and displayed on a management terminal.

### CITATION LIST

### Patent Literature

PTL 1: JP 2019-185408 A

### SUMMARY OF INVENTION

### Technical Problem

In the method of PTL 1, the degree of area health risk calculated for each area can be displayed in superimposition on a map. Therefore, according to the method of PTL 1, since the degree of the area health risk is displayed to be superimposed on the map, it is easy to grasp the health condition of the local residents. However, in the method of PTL 1, although the index of the individual health risk can be verified, the position of a person having a high risk of acquiring a specific disease cannot be visualized.

An object of the present disclosure is to provide an information generation device, an information provision system, an information generation method, and a recording medium capable of generating a risk map in which a position of a person at risk of acquiring a target disease is visualized.

### Solution to Problem

An information generation device according to one aspect of the present disclosure includes an acquisition unit that acquires sensor data measured by a measurement device mounted on footwear of at least one subject, a risk estimation unit that estimates a disease risk for each disease related to the at least one subject using the acquired sensor data, a map generation unit that generates a risk map in which a display according to a disease risk of a target disease related to the at least one subject is superimposed on a map of a target district, and an output unit that outputs risk information including the generated risk map.

In an information generation device according to an aspect of the present disclosure, sensor data measured by a measurement device mounted on footwear of at least one subject is acquired, a disease risk for each disease related to the at least one subject is estimated using the acquired sensor data, a risk map is generated in which a display according to a disease risk of a target disease related to the at least one subject is superimposed on a map of a target district, and risk information including the generated risk map is output.

A program according to an aspect of the present disclosure causes a computer to execute a process of acquiring sensor data measured by a measurement device mounted on footwear of at least one subject, a process of estimating a disease risk for each disease related to the at least one subject using the acquired sensor data, a process of generating a risk map in which a display according to a disease risk of a target disease related to the at least one subject is superimposed on a map of a target district, and a process of outputting risk information including the generated risk map.

### Advantageous Effects of Invention

According to the present disclosure, it is possible to provide an information generation device, an information provision system, an information generation method, and a recording medium capable of generating a risk map in which a position of a person at risk of acquiring a target disease is visualized.

### BRIEF DESCRIPTION OF DRAWINGS

FIG. 1 is a block diagram illustrating an example of a configuration of an information provision system in the present disclosure.

FIG. 2 is a block diagram illustrating an example of a configuration of a measurement device included in the information provision system in the present disclosure.

FIG. 3 is a conceptual diagram illustrating an arrangement example of a measurement device included in the information provision system in the present disclosure.

FIG. 4 is a conceptual diagram for explaining a coordinate system set in a measurement device included in the information provision system in the present disclosure.

FIG. 5 is a conceptual diagram for explaining a human body surface used in the description of the present disclosure.

FIG. 6 is a block diagram illustrating an example of a configuration of an information generation device included in the information provision system in the present disclosure.

FIG. 7 is a conceptual diagram for explaining a gait cycle used in the description of the present disclosure.

FIG. 8 is a conceptual diagram for explaining a physical ability estimation model used by the information generation device included in the information provision system in the present disclosure.

FIG. 9 is a conceptual diagram for explaining an example of estimation of a disease risk by the information provision system in the present disclosure.

FIG. 10 is a conceptual diagram for explaining an example of estimation of a disease risk by the information provision system in the present disclosure.

FIG. 11 is a conceptual diagram illustrating an example of a map of a target district used for generating a risk map by the information generation device included in the information provision system in the present disclosure.

FIG. 12 is a conceptual diagram illustrating an example of a risk map generated by the information generation device included in the information provision system in the present disclosure.

FIG. 13 is a conceptual diagram illustrating an example of a risk map generated by the information generation device included in the information provision system in the present disclosure.

FIG. 14 is a conceptual diagram illustrating an example of a risk map generated by the information generation device included in the information provision system in the present disclosure.

FIG. 15 is a conceptual diagram illustrating an example of a risk map generated by the information generation device included in the information provision system in the present disclosure.

FIG. 16 is a conceptual diagram illustrating an example of a risk map generated by the information generation device included in the information provision system in the present disclosure.

FIG. 17 is a flowchart for explaining an example of the operation of the information generation device included in the information provision system in the present disclosure.

FIG. 18 is a flowchart for explaining an example of gait index calculation processing by the information generation device included in the information provision system in the present disclosure.

FIG. 19 is a flowchart for explaining an example of risk map generation processing by the information generation device included in the information provision system in the present disclosure.

FIG. 20 is a conceptual diagram for explaining a service using the information provision system in the present disclosure.

FIG. 21 is a conceptual diagram illustrating a display example of a risk map provided from the information provision system in the present disclosure.

FIG. 22 is a block diagram illustrating an example of a configuration of the information provision system in the present disclosure.

FIG. 23 is a conceptual diagram illustrating an example of a configuration of the information generation device included in the information provision system in the present disclosure.

FIG. 24 is a conceptual diagram for explaining an example of estimation of measures by the information provision system in the present disclosure.

FIG. 25 is a flowchart for explaining an example of the operation of the information generation device included in the information provision system in the present disclosure.

FIG. 26 is a conceptual diagram illustrating a display example of a measure proposal provided from the information provision system in the present disclosure.

FIG. 27 is a block diagram illustrating an example of a configuration of the information generation device in the present disclosure.

FIG. 28 is a flowchart for explaining an example of the operation of the information generation device in the present disclosure.

FIG. 29 is a conceptual diagram illustrating an example of a hardware configuration in the present disclosure.

### EXAMPLE EMBODIMENT

Hereinafter, modes for carrying out the present disclosure will be described with reference to the drawings. In the present disclosure, the drawings used in description of each example embodiment are associated with one or more example embodiments. Elements included in each drawing may apply to one or more example embodiments. The example embodiments described below may have technical limitations for carrying out the present disclosure, but the scope of the disclosure is not limited to the following. In all the drawings used in the following description of the example embodiments, the same reference signs are given to similar parts unless otherwise specified. In the following example embodiments, repeated description of similar configurations and operations may be omitted. The directions of the arrows in the drawings illustrate an example, and do not limit the directions of data, signals, and the like.

### First Example Embodiment

First, an example of an information provision system in the present disclosure will be described with reference to the drawings. The information provision system according to the present example embodiment estimates a risk of acquiring a specific disease (also referred to as a disease risk) using sensor data related to movement of a foot according to the gait of a subject located in a target district for which a risk map is generated. The information provision system of the present example embodiment generates a risk map in which the distribution and position of people having a high disease risk are visualized. In the present example embodiment, an example in which a risk map in which disease risk for each disease is visualized is generated will be described.

### (Configuration)

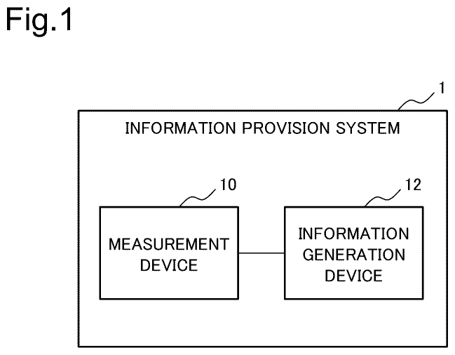

FIG. 1 is a block diagram illustrating an example of a configuration of an information provision system 1 in the present disclosure. The information provision system 1 includes a measurement device 10 and an information generation device 12. For example, the measurement device 10 is installed on the footwear of the subject who is an estimation target of a disease risk. For example, the function of the information generation device 12 is installed in a mobile terminal carried by the subject. For example, the function of the information generation device 12 is installed in a server or a cloud connected to a mobile terminal carried by the subject via a network. Hereinafter, configurations of the measurement device 10 and the information generation device 12 will be individually described.

### [Measurement Device]

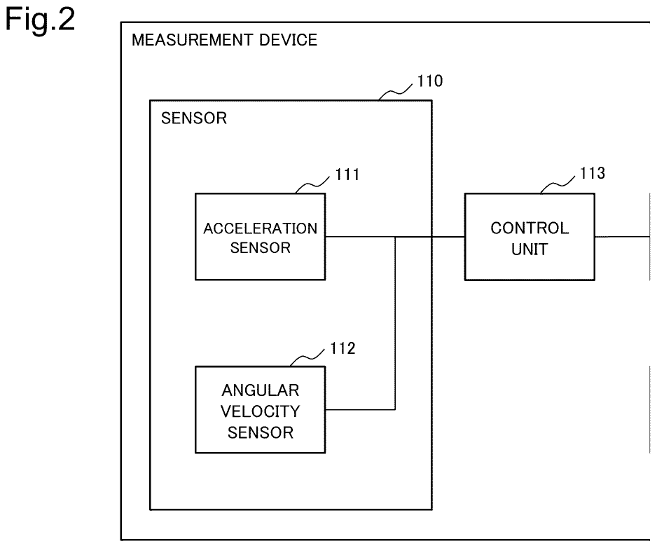

FIG. 2 is a block diagram illustrating an example of a configuration of the measurement device 10. The measurement device 10 includes a sensor 110, a control unit 113, a communication unit 115, and a power supply 117. The sensor 110 includes an acceleration sensor 111 and an angular velocity sensor 112. The sensor 110 may include a sensor other than the acceleration sensor 111 and the angular velocity sensor 112. Sensors other than the acceleration sensor 111 and the angular velocity sensor 112 that can be included in the sensor 110 will not be described.

The acceleration sensor 111 is a sensor that measures accelerations (also referred to as spatial accelerations) in three axial directions. The acceleration sensor 111 measures acceleration (also referred to as spatial acceleration) as a physical quantity related to the movement of the foot. The acceleration sensor 111 outputs the measured acceleration to the control unit 113. For example, a sensor of a piezoelectric type, a piezoresistive type, a capacitance type, or the like can be used as the acceleration sensor 111. The sensor used as the acceleration sensor 111 is not limited as long as it can measure acceleration.

The angular velocity sensor 112 is a sensor that measures angular velocity (also referred to as a spatial angular velocity) around three axes. The angular velocity sensor 112 measures angular velocity (also referred to as a spatial angular velocity) as a physical quantity related to the movement of the foot. The angular velocity sensor 112 outputs the measured angular velocity to the control unit 113. For example, a sensor of a vibration type, a capacitance type, or the like can be used as the angular velocity sensor 112. The sensor used as the angular velocity sensor 112 is not limited as long as the sensor can measure the angular velocity.

The sensor 110 is implemented by, for example, an inertial measurement device that measures acceleration and angular velocity. An example of the inertial measurement device is an inertial measurement unit (IMU). The IMU includes an acceleration sensor 111 that measures acceleration in three axis directions and an angular velocity sensor 112 that measures the angular velocity around the three axes. The sensor 110 may be implemented by an inertial measurement device such as a vertical gyro (VG) or an attitude heading reference system (AHRS). The sensor 110 may be implemented by GPS/INS (Global Positioning System/Inertial Navigation System). The sensor 110 may be implemented by a device other than the inertial measurement device as long as it can measure a physical quantity related to the movement of the foot.

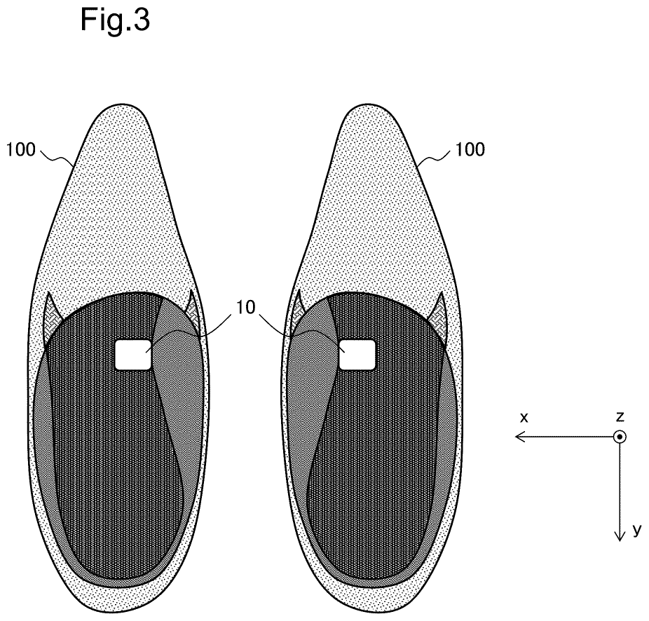

FIG. 3 is a conceptual diagram illustrating an example in which the measurement device 10 is disposed in shoes 100 of both feet. In the example of FIG. 3, the measurement device 10 is installed at a position relevant to the back side of the arch of a foot. For example, the measurement device 10 is disposed in an insole inserted into the shoe 100. For example, the measurement device 10 may be disposed on the bottom surface of the shoe 100. For example, the measurement device 10 may be embedded in the main body of the shoe 100. The measurement device 10 may be detachable from the shoe 100 or may not be detachable from the shoe 100. The measurement device 10 may be installed at a position other than the back side of the arch of the foot as long as the sensor data related to the movement of the foot can be measured. The measurement device 10 may be installed on a sock worn by the user or a decorative article such as an anklet worn by the user. The measurement device 10 may be directly attached to the foot or may be embedded in the foot. As long as data from which the disease risk can be estimated can be measured, the measurement device 10 may be disposed in one shoe 100.

In the example of FIG. 3, a local coordinate system including an x axis in the left-right direction, a y axis in the front-rear direction, and a z axis in the upward-downward direction is set with reference to the measurement device 10 (sensor 110). FIG. 3 illustrates an example in which the same coordinate system is set for the left foot and the right foot. For example, in a case where the sensors 110 produced with the same specifications are disposed in the left and right shoes 100, the upward-downward direction (directions in the Z-axis direction) of the sensors 110 disposed in the left and right shoes 100 are the same. In this case, the three axes of the local coordinate system set in the sensor data derived from the left foot and the three axes of the local coordinate system set in the sensor data derived from the right foot are the same on the left and right. In the present disclosure, the left side of the x axis is positive, the rear side of the y axis is positive, and the upper side of the z axis is positive.

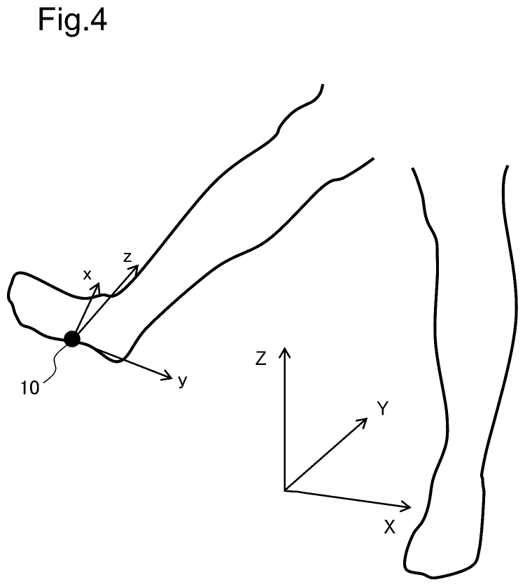

FIG. 4 is a conceptual diagram for explaining a local coordinate system (x axis, y axis, z axis) set in the measurement device 10 (sensor 110) installed on the back side of the arch of the foot and a world coordinate system (X axis, Y axis, Z axis) set with respect to the ground. FIG. 4 illustrates an example in which different coordinate systems are set for the left foot and the right foot. In the world coordinate system (X axis, Y axis, Z axis), the lateral direction of the user is set to the X-axis direction, the direction of the back surface of the user is set to the Y-axis direction, and the gravity direction is set to the Z-axis direction in a state where the user facing the traveling direction is upright. The example of FIG. 4 conceptually illustrates the relationship between the local coordinate system (x axis, y axis, z axis) and the world coordinate system (X axis, Y axis, Z axis), and does not accurately illustrate the relationship between the local coordinate system and the world coordinate system that varies depending on the gait of the user.

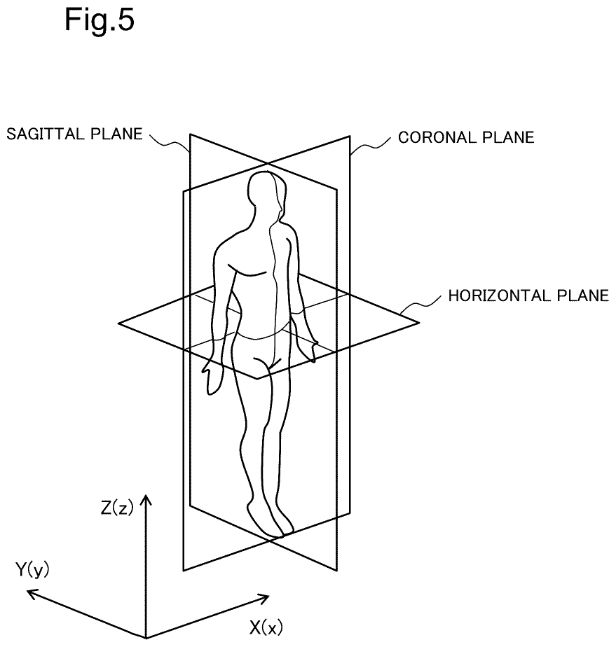

FIG. 5 is a conceptual diagram for explaining a surface (also referred to as a human body surface) set for the human body. In the present example embodiment, a sagittal plane dividing the body into left and right, a coronal plane dividing the body into front and rear, and a horizontal plane dividing the body horizontally are defined. As illustrated in FIG. 5, it is assumed that the world coordinate system and the local coordinate system coincide with each other in a state in which the center line of the foot is oriented in the traveling direction. FIG. 5 illustrates an example in which different coordinate systems are set for the left foot and the right foot. In the present example embodiment, rotation in a sagittal plane with the X axis (x axis) as a rotation axis is defined as a roll, rotation in a coronal plane with the Y axis (y axis) as a rotation axis is defined as a pitch, and rotation in a horizontal plane with the Z axis (z axis) as a rotation axis is defined as a yaw. A rotation angle in the sagittal plane with the X axis (x axis) as a rotation axis is defined as a roll angle, a rotation angle in the coronal plane with the Y axis (y axis) as a rotation axis is defined as a pitch angle, and a rotation angle in a horizontal plane with the Z axis (z axis) as a rotation axis is defined as a yaw angle.

The control unit 113 (control means) causes the acceleration sensor 111 and the angular velocity sensor 112 to measure sensor data. For example, the control unit 113 causes the acceleration sensor 111 and the angular velocity sensor 112 to start measurement in response to a measurement start signal transmitted from the information generation device 12. For example, the control unit 113 may cause the acceleration sensor 111 and the angular velocity sensor 112 to start measurement in response to detection of the gait of the user. For example, after the heights of both legs/feet in the vertical direction are the same over a predetermined period set in advance, the control unit 113 starts the measurement of the step width from a time point at which movement of one of the right and left feet in the traveling direction is detected as a starting point. The control unit 113 may be configured to start the measurement of the step width at a predetermined timing set in advance.

The control unit 113 acquires accelerations in three axial directions from the acceleration sensor 111. The control unit 113 acquires the angular velocity around three axes from the angular velocity sensor 112. For example, the control unit 113 performs analog-to-digital conversion (AD conversion) on the acquired physical quantities (analog data) such as angular velocity and acceleration. The physical quantities (analog data) measured by the acceleration sensor 111 and the angular velocity sensor 112 may be converted into digital data in each of the acceleration sensor 111 and the angular velocity sensor 112. For example, an AD conversion circuit that performs AD conversion on physical quantities (analog data) such as angular velocity and acceleration may be provided. The control unit 113 outputs the converted digital data (also referred to as sensor data) to the communication unit 115. For example, the control unit 113 may temporarily store the sensor data in a storage unit (not illustrated).

The sensor data includes at least acceleration data converted into digital data and angular velocity data converted into digital data. The acceleration data includes acceleration vectors in three axial directions. The angular velocity data includes angular velocity vectors around three axes. The acceleration data and the angular velocity data are associated with acquisition times of the data. The control unit 113 may add corrections such as mounting error correction, temperature correction, and linearity correction to the acceleration data and the angular velocity data.

For example, the control unit 113 may calculate at least one of gait indexes to be described later. In that case, the measurement device 10 outputs the calculated gait index to the information generation device 12. For example, the control unit 113 may calculate a feature quantity used for estimation of physical ability described later. In that case, the measurement device 10 outputs the calculated feature quantity to the information generation device 12.

For example, the control unit 113 is implemented by a microcomputer or a microcontroller that performs overall control and data processing of the measurement device 10. For example, the control unit 113 includes a central processing unit (CPU), a random access memory (RAM), a read only memory (ROM), a flash memory, and the like.

The communication unit 115 (communication means) acquires sensor data from the control unit 113. The communication unit 115 transmits the acquired sensor data to the information generation device 12. The sensor data transmitted from the communication unit 115 is received by the information generation device 12. The transmission timing of the sensor data is not particularly limited. For example, the communication unit 115 transmits sensor data at a preset transmission timing. For example, the communication unit 115 transmits the sensor data in real time in response to the measurement of the sensor data. For example, the communication unit 115 may store sensor data measured during a predetermined period and collectively transmit the stored sensor data at a preset timing. For example, the communication unit 115 (communication means) may be configured to receive the measurement start signal from the information generation device 12. In this case, the communication unit 115 outputs the received measurement start signal to the control unit 113.

For example, the communication unit 115 transmits sensor data to the information generation device 12 via wireless communication. For example, the communication unit 115 transmits sensor data to the information generation device 12 via a wireless communication function (not illustrated) conforming to a standard such as Bluetooth (registered trademark) or WiFi (registered trademark). The communication function of the communication unit 115 may conform to a standard other than Bluetooth (registered trademark) or WiFi (registered trademark). The communication unit 115 may transmit the sensor data to the information generation device 12 via a wire such as a cable.

The power supply 117 is a battery that supplies power for the measurement device 10 to operate. For example, the power supply 117 is implemented by a thin battery such as a coin type or a button type. For example, the power supply 117 is implemented by a primary battery such as a lithium primary battery, a silver oxide battery, an alkaline button battery, or an air zinc battery. In the case of being implemented by the primary battery, the power supply 117 may be implemented by a long-life battery. The power supply 117 may be implemented by a rechargeable secondary battery. In the case of being implemented by the secondary battery, the power supply 117 may be a battery that can be charged in a wired manner or may be a battery that can be charged wirelessly. When the power supply 117 can wirelessly supply power, the wireless power supply device may be disposed at a place where footwear is placed, such as an entrance or a footwear box. If the footwear on which the measurement device 10 is mounted is stacked on the wireless power supply device, the measurement device 10 can be charged appropriately when not in use.

### [Information Generation Device]

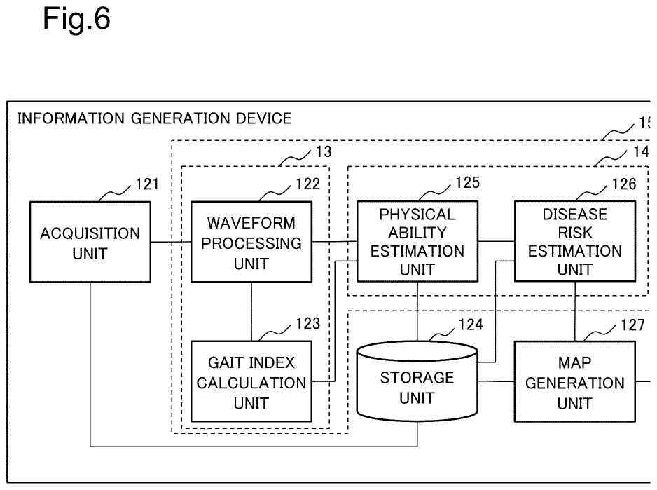

FIG. 6 is a block diagram illustrating an example of a configuration of the information generation device 12. The information generation device 12 includes an acquisition unit 121, a waveform processing unit 122, a gait index calculation unit 123, a storage unit 124, a physical ability estimation unit 125, a disease risk estimation unit 126, a map generation unit 127, and an output unit 129. The waveform processing unit 122, the gait index calculation unit 123, the physical ability estimation unit 125, and the disease risk estimation unit 126 constitute a risk estimation unit 15. The waveform processing unit 122 and the gait index calculation unit 123 constitute a calculation unit 13. The physical ability estimation unit 125 and the disease risk estimation unit 126 constitute an estimation unit 14.

The acquisition unit 121 (acquisition means) acquires sensor data from the measurement device 10 mounted on the footwear of the subject who uses the information provision system 1. The acquisition unit 121 receives sensor data from the measurement device 10 via wireless communication. The sensor data includes position information of a mobile terminal (not illustrated) of the subject who is a transmission source of the sensor data. For example, the position information is measured by a function of a global positioning system (GPS) mounted on the mobile terminal. For example, the acquisition unit 121 receives sensor data from the measurement device 10 via a wireless communication function (not illustrated) conforming to a standard such as Bluetooth (registered trademark) or WiFi (registered trademark). The communication function of the acquisition unit 121 may conform to a standard other than Bluetooth (registered trademark) or WiFi (registered trademark) as long as the communication function can communicate with the measurement device 10. The acquisition unit 121 may receive the sensor data from the measurement device 10 via a wire such as a cable. For example, the acquisition unit 121 may acquire a gait index or a feature quantity calculated by the measurement device 10.

The acquisition unit 121 acquires attribute data of the subject. The attribute data includes gender, date of birth, height, and weight. The date of birth is converted to age. The gender, the date of birth (age), the height, and the weight included in the attribute data are also referred to as body information. The attribute data includes the address (position information) of the residence of the subject. The address (position information) of the residence of the subject is used to generate the risk map of the target district. Usually, the address (position information) of the residence of the subject is not used for estimating the physical ability or the disease risk. For example, the attribute data is input via an input device (not illustrated). For example, the attribute data is input via a mobile terminal used by the subject. For example, the attribute data may be stored in the storage unit 124 in advance. The attribute data may be updated at an arbitrary timing according to the input by the subject.

The waveform processing unit 122 (waveform processing means) acquires sensor data from the acquisition unit 121. The waveform processing unit 122 extracts time-series data for one gait cycle from the time-series data of the acceleration in the three-axis direction and the angular velocity around the three axes included in the sensor data. The time-series data for one gait cycle is also referred to as gait waveform data. The waveform processing unit 122 extracts the gait waveform data based on the timing of the gait event detected from the time-series data of the sensor data. For example, the waveform processing unit 122 extracts the gait waveform data with the timing of the heel strike as a start point and the timing of the next heel strike as an end point.

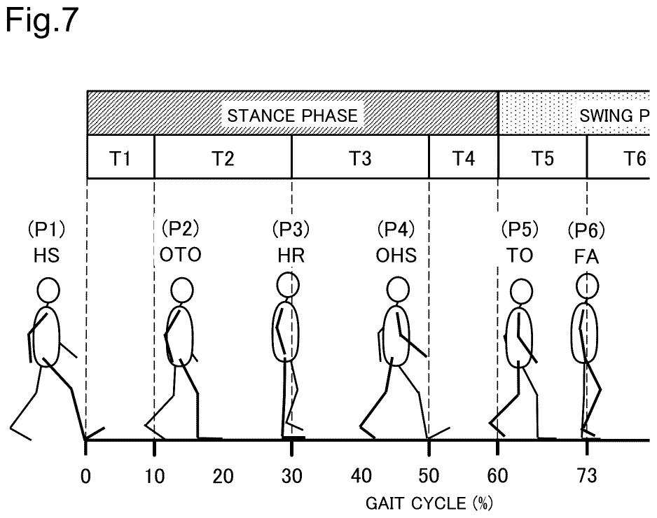

FIG. 7 is a conceptual diagram for explaining one gait cycle with the right foot as a reference. One gait cycle based on the left foot is also similar to that of the right foot. The horizontal axis in FIG. 7 indicates one gait cycle of the right foot with a time point at which the heel of the right foot lands on the ground as a starting point and a time point at which the heel of the right foot next lands on the ground as an ending point. The horizontal axis in FIG. 7 is normalized with one gait cycle as 100%. Normalizing one gait cycle by 100% is referred to as first normalization. The one gait cycle of one foot is roughly divided into a stance phase in which at least a part of the back side of the foot is in contact with the ground and a swing phase in which the back side of the foot is separated from the ground. The stance phase is a period in which at least a part of the back side of the foot is in contact with the ground. The stance phase is further subdivided into an initial stance period T1, a mid-stance period T2, a terminal stance period T3, and a pre-swing period T4. The swing phase is a period in which the back side of the foot is away from the ground. The swing phase is further subdivided into an initial swing period T5, a mid-swing period T6, and a terminal swing period T7. The horizontal axis in FIG. 7 is normalized such that the stance phase is 60% and the swing phase is 40%. The normalization of the gait waveform data so that the stance phase becomes 60% and the swing phase becomes 40% is referred to as second normalization. The period illustrated in FIG. 7 is an example, and the period constituting one gait cycle, the name of those periods, and the like are not limited.

As illustrated in FIG. 7, a plurality of events occur during the gait. In the gait, a plurality of events in the gait are also referred to as gait events. P1 represents an event (heel strike) in which the heel of the right foot touches the ground (HS: Heel Strike). P2 represents an event (opposite toe off) in which the toe of the left foot is separated from the ground in a state where the sole of the right foot is grounded (OTO: Opposite Toe Off). P3 represents an event (heel rise) in which the heel of the right foot lifts in a state where the sole of the right foot is grounded (HR: Heel Rise). P4 is an event (opposite heel strike) in which the heel of the left foot is grounded (OHS: Opposite Heel Strike). P5 represents an event (toe off) in which the toe of the right foot is separated from the ground in a state where the sole of the left foot is grounded (TO: Toe Off). P6 represents an event (foot adjacent) in which the left foot and the right foot cross each other in a state where the sole of the left foot is grounded (FA: Foot Adjacent). P7 represents an event (tibia vertical) in which the tibia of the right foot is approximately perpendicular to the ground in a state where the sole of the left foot is grounded (TV: Tibia Vertical). P8 represents an event (heel strike) in which the heel of the right foot touches the ground (HS: Heel Strike). P8 is relevant to the end point of the gait cycle starting from P1 and is relevant to the start point of the next gait cycle. The gait event illustrated in FIG. 7 is an example, and does not limit the events that occur during the gait or the names of these events.

The timing of the heel strike is the timing of a local minimum peak immediately after a local maximum peak appearing in the time-series data of the acceleration in the traveling direction (acceleration in the Y direction). The local maximum peak serving as a mark of the heel strike timing is relevant to the maximum peak of the gait waveform data for one gait cycle. A section between consecutive heel strikes is relevant to one gait cycle. The timing of the toe off is the rising timing of the local maximum peak appearing after the period of the stance phase in which the fluctuation does not appear in the time-series data of the acceleration in the traveling direction (acceleration in the Y direction). The timing at the midpoint between the timing at which the roll angle is minimum and the timing at which the roll angle is maximum is relevant to the mid-stance period.

The waveform processing unit 122 normalizes the time of the extracted gait waveform data for one gait cycle to a gait cycle of 0 to 100% (percent) (first normalization). Timing such as 1% or 10% included in the 0 to 100% gait cycle is also referred to as a gait phase. The waveform processing unit 122 normalizes the gait waveform data subjected to the first normalization for one gait cycle so that the stance phase becomes 60% and the swing phase becomes 40% (second normalization). By performing the second normalization on the gait waveform data, it is possible to reduce the shift of the gait phase from which the feature quantity is extracted. The waveform processing unit 122 outputs the normalized gait waveform data to the gait index calculation unit 123.

For example, the waveform processing unit 122 extracts/normalizes the gait waveform data for one gait cycle using the acceleration in the traveling direction (acceleration in the Y direction). With respect to acceleration/angular velocity other than the acceleration in the traveling direction (acceleration in the Y direction), the waveform processing unit 122 extracts/normalizes gait waveform data for one gait cycle in accordance with the gait cycle of the acceleration in the traveling direction (acceleration in the Y direction). The waveform processing unit 122 may generate time-series data of the angle around three axes by integrating time-series data of the angular velocity around the three axes. In that case, the waveform processing unit 122 also extracts/normalizes the gait waveform data for one gait cycle in accordance with the gait cycle of the acceleration in the traveling direction (acceleration in the Y direction) with respect to the angle around the three axes.

The waveform processing unit 122 may extract/normalize the gait waveform data for one gait cycle using acceleration/angular velocity other than the acceleration in the traveling direction (acceleration in the Y direction). For example, the waveform processing unit 122 may detect the heel strike and the toe off from the time-series data of the vertical acceleration (acceleration in the Z direction) (omitted in the drawing). The timing of the heel strike is a timing of a steep local minimum peak appearing in the time-series data of the vertical acceleration (acceleration in the Z direction). At the timing of the steep local minimum peak, the value of the vertical acceleration (acceleration in the Z direction) becomes substantially zero. The local minimum peak serving as a mark of the timing of the heel strike is relevant to the minimum peak of the gait waveform data for one gait cycle. A section between consecutive heel strikes is one gait cycle. The timing of the toe off point is a timing of an inflection point in the middle of gradually increasing after the time-series data of the acceleration in the vertical direction (acceleration in the Z direction) passes through a section with a small fluctuation after the local maximum peak immediately after the heel strike. The waveform processing unit 122 may extract/normalize the gait waveform data for one gait cycle using both the acceleration in the traveling direction (acceleration in the Y direction) and the acceleration in the vertical direction (acceleration in the Z direction). The waveform processing unit 122 may extract/normalize the gait waveform data for one gait cycle using acceleration, angular velocity, angle, and the like other than the acceleration in the traveling direction (acceleration in the Y direction) and the acceleration in the vertical direction (acceleration in the Z direction).

The waveform processing unit 122 extracts a feature quantity (physical ability feature quantity) used to estimate the physical ability from the gait waveform data. The waveform processing unit 122 extracts a physical ability feature quantity used to estimate at least one physical ability. For example, the waveform processing unit 122 extracts a physical ability feature quantity used for estimation of at least one of physical abilities such as grip strength (total muscle strength of the whole body), dynamic balance, lower limb muscle strength, movement ability, and static balance. For example, the waveform processing unit 122 extracts the physical ability feature quantity for each gait phase cluster according to a preset condition. The gait phase cluster is a cluster in which temporally continuous gait phases are integrated. The gait phase cluster includes at least one gait phase. The gait phase cluster also includes a single gait phase. The waveform processing unit 122 outputs the extracted physical ability feature quantity to the physical ability estimation unit 125.

The gait index calculation unit 123 (gait index calculation means) acquires the normalized gait waveform data from the waveform processing unit 122. The gait index calculation unit 123 calculates a gait index used for estimating the physical ability using the normalized gait waveform data. The gait index to be calculated is not particularly limited as long as the gait index can be calculated using the normalized gait waveform data. For example, the gait index calculation unit 123 calculates a gait index related to a distance, a height, an angle, a speed, a time, a frailty level, a center of pressure exclusion index (CPEI), and the like. Hereinafter, representative gait indexes will be described. A specific calculation method of the following gait index will be omitted.

For example, the gait index calculation unit 123 calculates an index related to a distance and a height as a gait index. For example, the gait index calculation unit 123 calculates a stride length, an outward turning distance, foot raising height, a foot clearance (FTC), and a minimum toe clearance (MTC). The stride length indicates a distance between a front foot and a rear foot during the gait. The outward turning distance indicates the maximum value of the distance at which the foot is separated outward with respect to the traveling direction in the swing phase. The foot raising height indicates the maximum value of the distance between the measurement device 10 (sensor 110) and the ground in the swing phase. The FTC indicates the maximum value of the distance between the heel and the ground in the swing phase. The MTC indicates the minimum value of the distance between the toe and the ground in the swing phase.

For example, the gait index calculation unit 123 calculates an index related to an angle as the gait index. For example, the gait index calculation unit 123 calculates the grounding angle, the ground separation angle, the toe direction, the roll angle of the heel strike, the roll angle of the toe off, the swing peak angular velocity, and the hallux angle. The grounding angle indicates a maximum value of an angle formed by the sole surface and the ground at the time of heel strike. The ground separation angle indicates an angle formed between the sole surface and the ground in the swing phase. The toe direction indicates an average value of the toe directions with respect to the traveling direction in the swing phase. The roll angle of the heel strike is an angle formed between the ankle and the ground at the time of the heel strike when viewed from the rear viewing seat. The roll angle of the toe off ground is an angle formed between the ankle and the ground at the time of kicking as viewed from the rear viewing seat. The swing peak angular velocity is the angular velocity in the ankle joint dorsiflexion direction in a section from immediately after kicking until the toe comes into closest contact with the ground. The hallux angle indicates an angle at which the thumb of the foot is inclined toward the index finger. Specifically, the hallux angle is an angle formed by the center line of the first metatarsal and the center line of the first proximal phalanx.

For example, the gait index calculation unit 123 calculates an index related to a speed as the gait index. For example, the gait index calculation unit 123 calculates a gait speed, cadence, and a maximum speed in swing. The gait speed indicates a speed in the gait. The cadence indicates the number of steps per minute. The maximum speed in swing indicates a speed at which the user swings out the leg in the swing phase.

For example, the gait index calculation unit 123 calculates an index related to a time as the gait index. For example, the gait index calculation unit 123 calculates a standing time, a load time, a plantar grounding time, a kicking time, a swing time, and a double support time (DST). The standing time indicates a time during which the foot is grounded during the gait. The standing time is a sum of the load time, the plantar grounding time, and the kicking time. The load time is a time from when the heel is grounded to when the toe is grounded in the stance phase. The plantar grounding time is a time during which the entire plantar surface is grounded and the plantar surface and the ground are horizontal in the stance phase. The kicking time is a time until the toe kicks the ground from the state of the sole grounding in the stance phase. The swing time indicates a time during which the foot is separated from the ground during the gait. The DST is divided into DST1 and DST2. DST1 indicates a time during which the foot on which the measurement device 10 (sensor 110) is mounted is in front of the opposite foot in a period in which both feet are simultaneously grounded. DST2 indicates a time during which the foot on which the measurement device 10 (sensor 110) is mounted is behind the opposite foot in a period in which both feet are simultaneously grounded.

For example, the gait index calculation unit 123 calculates a frailty level or a center of pressure exclusion index (CPEI) as the gait index. The frailty level is an estimated value of the frail state according to the gait state. For example, the gait index calculation unit 123 estimates indexes such as a determination result indicating health, a determination result indicating a possibility of frailty, and a determination result having a high possibility of frailty as the frailty level. The CPEI indicates an estimated value of the expansion ratio of the movement of the center of foot pressure applied to the ground during the stance phase.

The storage unit 124 (storage means) stores a physical ability estimation model (described later) for estimating the physical ability using the physical ability feature quantity extracted from the gait waveform data. For example, the physical ability is at least one of grip strength, dynamic balance, lower limb muscle strength, movement ability, and static balance. The physical ability may include other than grip strength, dynamic balance, lower limb muscle strength, movement ability, and static balance. The storage unit 124 stores a physical ability estimation model learned for a plurality of subjects. For example, the physical ability estimation model outputs an index (physical ability score) related to the physical ability according to the input of the physical ability feature quantity extracted from the gait waveform data.

The storage unit 124 stores a disease risk estimation model (described later) for estimating a disease risk using the attribute data, the gait index, and the physical ability score. The disease risk indicates a risk of acquiring a specific disease. For example, the specific diseases include gout, diabetes, hypertension, nephrolithiasis, liver cirrhosis, arteriosclerosis, thromboembolism, dyslipidemia, hypercholesterolemia, and hyperlipidemia. For example, the specific diseases include low back pain, sleep apnea syndrome, insomnia, depression, knee osteoarthritis, and Parkinson's syndrome. The specific disease may include diseases other than those described above. The storage unit 124 stores a disease risk estimation model learned for a plurality of subjects. For example, the disease risk estimation model outputs an index related to the disease risk (disease risk score) according to the input of the attribute data, the gait index, and the physical ability score. Usually, the address (position information) of the residence of the subject included in the attribute data is not used for estimating the physical ability or the disease risk. For example, the disease risk estimation model may be a model that outputs the disease risk score according to the input of the gait index and the attribute data without using the physical ability score. In that case, the physical ability estimation model may not be used.

For example, the physical ability estimation model and the disease risk estimation model may be stored in the storage unit 124 at the time of factory shipment of a product. The physical ability estimation model and the disease risk estimation model may be stored in the storage unit 124 at the timing of calibration before the subject uses the information generation device 12. For example, a physical ability estimation model and a disease risk estimation model stored in a storage device (not illustrated) such as an external server may be used. In that case, it is sufficient that the physical ability estimation model and the disease risk estimation model can be accessed via an interface (not illustrated) connected to the storage device.

The storage unit 124 stores the attribute of the subject. The attribute data includes gender, date of birth (age), height, and weight. The attribute data includes the address (position information) of the residence of the subject. Usually, the address (position information) of the residence of the subject is not used for estimating the physical ability or the disease risk. The attribute data may be updated at an arbitrary timing.

Furthermore, the storage unit 124 stores a map of a target district for which the risk map is generated. The map of the target district may be stored in advance in the storage unit 124. For example, the map of the target district may not be stored in the storage unit 124 and may be acquired from an external database by the acquisition unit 121.

The physical ability estimation unit 125 (physical ability estimation means) acquires the physical ability feature quantity extracted from the gait waveform data from the waveform processing unit 122. The physical ability estimation unit 125 acquires the attributes stored in the storage unit 124. The physical ability estimation unit 125 estimates the physical ability score using the physical ability feature quantity and the attribute. The physical ability estimation unit 125 inputs the physical ability feature quantity and the attribute of the subject to the physical ability estimation model stored in the storage unit 124. For example, the physical ability estimation unit 125 estimates a physical ability score related to the physical ability of at least one of grip strength (total muscle strength of the whole body), dynamic balance, lower limb muscle strength, movement ability, or static balance. The estimation of the physical ability score by the physical ability estimation unit 125 will be described later. The physical ability estimation unit 125 outputs the physical ability score output from the physical ability estimation model to the disease risk estimation unit 126.

Next, an estimation example of the physical ability score by the physical ability estimation unit 125 will be described with an example. Here, an example of feature quantities used for estimation of grip strength (total muscle strength of the whole body), dynamic balance, lower limb muscle strength, movement ability, and static balance will be described. The following examples do not limit the physical ability estimated by the physical ability estimation unit 125. The physical ability estimated by the physical ability estimation unit 125 may be appropriately selected according to a disease for which a disease risk is to be estimated. The disease risk estimation unit 126 may be configured to estimate the disease risk using the gait index and the attribute data without using the physical ability score. In that case, the physical ability estimation unit 125 may be omitted from the estimation unit 14.

### <Grip Strength (Total Muscle Strength of Whole Body)>

There is a correlation between grip strength, which is one of physical abilities, and total muscle strength of the whole body. The grip strength is also correlated with the knee extension strength. For example, the estimated value of the grip strength is an index of the total muscle strength. For example, a score relevant to the estimated value of the grip strength (also referred to as a total muscle strength score) is an index of the total muscle strength. The total muscle strength score is a value obtained by scoring the grip strength, which is an index of the total muscle strength, on a preset basis. The grip strength is affected by attributes such as gender, age, and height. Therefore, the total muscle strength score may be scored based on a criterion for each attribute. In particular, the grip strength is affected by gender. Therefore, the total muscle strength score may be scored based on different criteria depending on the gender. The index of the total muscle strength is not limited to the grip strength as long as the total muscle strength can be scored.

The gait phase in which the feature quantity used to estimate the grip strength is extracted differs depending on the gender. For male, there is a correlation between the activities of quadriceps muscles and the grip strength. Therefore, in the estimation of the grip strength of the male, the feature quantity extracted from the gait phase in which the feature of the activities of quadriceps muscles appears is used. For female, there is a correlation between the activities of the Vastus lateralis, the Vastus intermedius, and the Vastus medialis of the quadriceps muscles and the grip strength. Therefore, in the estimation of the grip strength of a female, the feature quantity extracted from the gait phase in which the features of the activities of the Vastus lateralis, the Vastus intermedius, and the Vastus medialis appear is used.

A feature quantity AM1, a feature quantity AM2, a feature quantity AM3, and a feature quantity AM4 are used to estimate the grip strength of a male. The feature quantity AM1 is extracted from the section of the gait phase 3% of the gait waveform data related to the time-series data of the acceleration in the traveling direction (acceleration in the Y direction). The gait phase 3% is included in the initial stance period T1.

The feature quantity AM1 mainly includes features related to the movements of the Vastus lateralis, the Vastus intermedius, and the Vastus medialis of the quadriceps muscles. The feature quantity AM2 is extracted from the section of the gait phase 59 to 62% of the gait waveform data related to the time-series data of the acceleration in the traveling direction (acceleration in the Y direction). The gait phase 59 to 62% is included in the pre-swing period T4. The feature quantity AM2 mainly includes a feature related to movement of the rectus femoris muscle of the quadriceps muscles. The feature quantity AM3 is extracted from the section of the gait phase 59 to 62% of the gait waveform data related to the time-series data of the vertical acceleration (acceleration in the Z direction). The gait phase 59 to 62% is included in the pre-swing period T4. The feature quantity AM3 mainly includes a feature related to movement of the rectus femoris muscle of the quadriceps muscles. The feature quantity AM4 is a ratio (DST1) of a period from the heel strike to the opposite toe off in the period in which both feet are simultaneously grounded. DST1 is a ratio of a period from the heel strike to the opposite toe off in one gait cycle. The feature quantity AM4 mainly includes a feature caused by the quadriceps muscles.

A feature quantity AF1, a feature quantity AF2, and a feature quantity AF3 are used to estimate the grip strength of a female. The feature quantity AF1 is extracted from a section of the gait phase 13% of the gait waveform data related to the time-series data of the acceleration in the lateral direction (acceleration in the X direction). The gait phase 13% is included in the mid-stance period T2. The feature quantity AF1 mainly includes features related to the movements of the Vastus lateralis, the Vastus intermedius, and the Vastus medialis of the quadriceps muscles. The feature quantity AF2 is extracted from a section of the gait phase 7 to 10% of the gait waveform data related to the time-series data of the angular velocity (pitch angular velocity) in the coronal plane (around the Y axis). The gait phase 7 to 10% is included in the initial stance period T1. The feature quantity AF2 mainly includes features related to movements of the Vastus lateralis, the Vastus intermedius, and the Vastus medialis. The feature quantity AF3 is a ratio (DST2) of a period from the opposite heel strike to the toe off in the period in which both feet are simultaneously grounded. DST2 is a ratio of a period from the opposite heel strike to the toe off in one gait cycle. The sum of DST1 and DST2 is relevant to a period in which both feet are simultaneously grounded in one gait cycle. The feature quantity AF3 mainly includes features related to movements of the Vastus lateralis, the Vastus intermedius, and the Vastus medialis.

### <Dynamic Balance>

Dynamic balance, one of physical abilities, can be evaluated by the performance of a functional reach test (FRT). In the present disclosure, the performance of the FRT is evaluated by the distance between the fingertips (also referred to as functional reach distance) in a state where both hands are raised 90 degrees with respect to the horizontal plane and the upper limb is moved forward as much as possible. The functional reach distance (hereinafter, referred to as an FR distance) is a performance value of the FRT. The larger the FR distance, the higher the FRT performance. Dynamic balance may be assessed outside of the FRT performed with both hands. For example, dynamic balance may be assessed in terms of performance on one-handed FRT or other variations of the FRT.

An index of the dynamic balance is an FR distance. For example, an estimated value of the FR distance is an index of the dynamic balance. For example, a score relevant to the estimated value of the FR distance (also referred to as a dynamic balance score) is an index of the dynamic balance. The dynamic balance score is a value obtained by scoring an FR distance, which is an index of the dynamic balance, based on a preset criterion. The dynamic balance is affected by attributes such as height. Therefore, the dynamic balance score may be scored based on a criterion for each attribute. The index of the dynamic balance is not limited to the FR distance as long as the dynamic balance can be scored. The FR distance is correlated with the activities of the gluteus medius, the iliacus muscle, the hamstrings (the long head of the biceps femoris), the tibialis anterior, and the like, and the magnitude of the compensatory motion that makes the orientation of the toe outward. Therefore, feature quantities extracted from the gait phase in which these features appear are used to estimate the FR distance.

A feature quantity B1, a feature quantity B2, a feature quantity B3, a feature quantity B4, and a feature quantity B5 are used to estimate the FR distance. The feature quantity B1 is extracted from the section of the gait phase 75 to 79% of the gait waveform data related to the time-series data of the acceleration in the traveling direction (acceleration in the Y direction). The gait phase 75 to 79% is included in the mid-swing period T6. The feature quantity B1 mainly includes a feature related to movement of the tibialis anterior and the short head of the biceps femoris. The feature quantity B2 is extracted from the section of the gait phase 62% of the gait waveform data related to the time-series data of the vertical acceleration (acceleration in the Z direction). The gait phase 62% is included in the initial swing period T5. The feature quantity B2 mainly includes a feature related to movement of the iliacus muscle. The feature quantity B3 is extracted from the section of the gait phase 7 to 8% of the gait waveform data related to the time-series data of the angular velocity in the coronal plane (around the Y axis). The gait phase 7 to 8% is included in the initial stance period T1. The feature quantity B3 mainly includes a feature related to the movement of the gluteus medius. The feature quantity B4 is extracted from the section of the gait phase 57 to 58% of the gait waveform data related to the time-series data of the angle (posture angle) in the horizontal plane (around the Z axis). The gait phase 57 to 58% is included in the pre-swing period T4. The feature quantity B4 mainly includes a feature related to a compensatory motion. The compensatory motion is a motion of changing the foot angle to acquire stability in order to compensate for a decrease in balance ability and muscle function associated with aging. The feature quantity B5 is an average value of the foot angles in the horizontal plane in the swing phase. For example, the feature quantity B5 is an average value in the swing phase of the gait waveform data. In other words, the feature quantity B5 is an integral value of the gait waveform data related to the time-series data of the angular velocity in the horizontal plane (around the Z axis). The feature quantity B5 mainly includes a feature related to a compensatory motion.

### <Lower Limb Muscle Strength>

Lower limb muscle strength, which is one of physical abilities, can be evaluated by the performance of a chair standing test. In the present disclosure, the performance of a five-time chair standing test in which standing and sitting from a chair is repeated five times is evaluated. The five-time chair standing test is also referred to as an SS-5 (Sit to Stand-5) test. The performance of the five-time chair standing test is evaluated by the time for repeating standing-up and sitting-down from a chair five times (also referred to as a sit and stand time). The sit and stand time is a performance value of the SS-5 test. The shorter the sit and stand time, the higher the performance in the SS-5 test. The performance may be evaluated by a score of a 30-second chair standing (CS-30) test for measuring the number of times of sit and stand motions from a chair in 30 seconds.

The index of lower limb muscle strength is a sit and stand time. For example, an estimated value of time taking for sitting and standing five times is an index of lower limb muscle strength. For example, a score relevant to the estimated value of the sit and stand time (also referred to as lower limb muscle strength score) is an index of lower limb muscle strength. The lower limb muscle strength score is a value obtained by scoring a sit and stand time, which is an index of lower limb muscle strength, based on a preset criterion. Lower limb muscle strength is affected by attributes such as age. Therefore, the lower limb muscle strength score may be scored based on a criterion for each attribute. The index of lower limb muscle strength is not limited to the sit and stand time as long as lower limb muscle strength can be scored. The sit and stand time is correlated with quadriceps muscles, hamstrings, tibialis anterior, and gastrocnemius muscle. Therefore, feature quantities extracted from the gait phase in which these features appear are used to estimate the sit and stand time.

The estimation of lower limb muscle strength includes a feature quantity C1, a feature quantity C2, a feature quantity C3, and a feature quantity C4. The feature quantity C1 is extracted from the section of the gait phase 42 to 54% of the gait waveform data related to the time-series data of the angular velocity in the sagittal plane (around the X axis). The gait phase 42 to 54% is a section from the terminal stance period T3 to the pre-swing period T4. The feature quantity C1 mainly includes a feature related to movement of the gastrocnemius muscle. The feature quantity C2 is extracted from the section of the gait phase 99 to 100% of the gait waveform data related to the time-series data of the angular velocity in the coronal plane (around the Y axis). The gait phase 99 to 100% is the end stage of the terminal swing period T7. The feature quantity C2 mainly includes features related to movement of the quadriceps muscles, hamstrings, and tibialis anterior. The feature quantity C3 is extracted from the section of the gait phase 10 to 12% of the gait waveform data related to the time-series data of the angular velocity in the coronal plane (around the Y axis). The gait phase 10 to 12% is the early stage of the mid-stance period T2. The feature quantity C3 mainly includes features related to movement of the quadriceps muscles, hamstrings, and gastrocnemius muscles. The feature quantity C4 is extracted from the section of the gait phase 99% of the gait waveform data related to the time-series data of the angle (posture angle) in the horizontal plane (around the Z axis). The gait phase 99% is the end stage of the terminal swing period T7. The feature quantity C4 mainly includes features related to movement of the quadriceps muscles, hamstrings, and tibialis anterior.

### <Movement Ability>

Movement ability, one of physical abilities, can be assessed by TUG (Time Up and Go) test performance. In the present disclosure, the performance of the TUG test is evaluated by the time (also referred to as TUG required time) from standing up from a chair, walking to a mark 3 m (meters) ahead to change the direction, and sitting down again on the chair. The TUG required time is a performance value of the TUG test. The shorter the TUG required time, the higher the TUG test performance. Movement ability may be assessed by test performance on movement ability other than the TUG test.

The index of the movement ability is the TUG required time. For example, the estimated value of the TUG required time is an index of the movement ability. For example, a score relevant to the estimated value of the TUG required time (also referred to as a movement ability score) is an index of the movement ability. The movement ability score is a value obtained by scoring the TUG required time, which is an index of the movement ability, based on a preset criterion. Movement ability is affected by attributes such as age. Therefore, the movement ability score may be scored based on a criterion for each attribute. The index of the movement ability is not limited to the TUG required time as long as the movement ability can be scored. The TUG required time is correlated with the quadriceps muscles, the gluteus medius, and the tibialis anterior muscle. Therefore, feature quantities extracted from the gait phase in which these features appear are used to estimate the TUG required time.

A feature quantity D1, a feature quantity D2, a feature quantity D3, a feature quantity D4, a feature quantity D5, and a feature quantity D6 are used to estimate the movement ability. The feature quantity D1 is extracted from a section of the gait phase 64 to 65% of the gait waveform data related to the time-series data of the acceleration in the lateral direction (acceleration in the X direction). The gait phase 64 to 65% is included in the initial swing period T5. The feature quantity D1 mainly includes a feature related to the motion of the quadriceps muscles in the sit and stand motion. The feature quantity D2 is extracted from the section of the gait phase 57 to 58% of the gait waveform data related to the time-series data of the angular velocity in the sagittal plane (around the X axis). The gait phase 57 to 58% is included in the pre-swing period T4. The feature quantity D2 mainly includes a feature related to the motion of the quadriceps muscles related to the leg kicking speed. The feature quantity D3 is extracted from a section of the gait phase 19 to 20% of the gait waveform data related to the time-series data of the angular velocity in the coronal plane (around the Y axis). The gait phase 19 to 20% is included in the mid-stance period T2. The feature quantity D3 mainly includes a feature related to the movement of the gluteus medius in the direction change. The feature quantity D4 is extracted from the section of the gait phase 12 to 13% of the gait waveform data related to the time-series data of the angular velocity in the horizontal plane (around the Z axis). The gait phase 12 to 13% is the early stage of the mid-stance period T2. The feature quantity D4 mainly includes a feature related to the movement of the gluteus medius in the direction change. The feature quantity D5 is extracted from the section of the gait phase 74 to 75% of the gait waveform data related to the time-series data of the angular velocity in the horizontal plane (around the Z axis). The gait phase 74 to 75% is the early stage of the mid-swing period T6. The feature quantity D5 mainly includes a feature related to the movement of the tibialis anterior in standing and sitting and direction change. The feature quantity D6 is extracted from the section of the gait phase 76 to 80% of the gait waveform data related to the time-series data of the angle (posture angle) in the coronal plane (around the Y axis). The gait phase 76 to 80% is included in the mid-swing period T6. The feature quantity D6 mainly includes a feature related to the movement of the tibialis anterior in standing and sitting and direction change.

### <Static Balance>

Static balance, one of physical abilities, can be assessed by the performance of a single-leg standing test. In the present disclosure, the performance of the single-leg standing test is evaluated for a time (also referred to as a single-leg standing time) during which the eyes are closed and one leg is kept raised from the ground by 5 cm (centimeter). The single-leg standing time is a performance value of static balance. The larger the single-leg standing time, the higher the static balance performance. Static balance may be assessed with a performance other than the closed-eyes single-leg standing test. For example, the static balance may be evaluated in a single-leg standing test with eyes open (open-eyes single-leg standing test) or other variations of the single-leg standing test.

The static balance index is the single-leg standing time. For example, the estimated value of the single-leg standing time is an index of the static balance. For example, a score relevant to the estimated value of the single-leg standing time (also referred to as a static balance score) is an index of the static balance. The static balance score is a value obtained by scoring a single-leg standing time which is an index of the static balance based on a preset criterion. The static balance is affected by attributes such as age and height. Therefore, the static balance score may be scored based on a criterion for each attribute. The index of the static balance is not limited to the single-leg standing time as long as the static balance can be scored. The single-leg standing time is correlated with the gluteus medius, the adductor longus, the sartorius, and the adductor muscles. Therefore, the feature quantity extracted from the gait phase in which these features appear is used to estimate the single-leg standing time.

For the estimation of the static balance, a feature quantity E1, a feature quantity E2, a feature quantity E3, a feature quantity E4, a feature quantity E5, a feature quantity E6, and a feature quantity E7 are used. The feature quantity E1 is extracted from a section of the gait phase 13 to 19% of the gait waveform data related to the time-series data of the acceleration in the lateral direction (acceleration in the X direction). The gait phase 13 to 19% is included in the mid-stance period T2. The feature quantity E1 mainly includes a feature related to the movement of the gluteus medius. The feature quantity E2 is extracted from the section of the gait phase 95% of the gait waveform data related to the time-series data of the vertical acceleration (acceleration in the Z direction). The gait phase 95% is the end stage of the terminal swing period T7. The feature quantity E2 mainly includes a feature related to the movement of the gluteus medius. The feature quantity E3 is extracted from a section of the gait phase 64 to 65% of the gait waveform data related to the time-series data of the angular velocity in the coronal plane (around the Y axis). The gait phase 64 to 65% is included in the initial swing period T5. The feature quantity E3 mainly includes a feature related to movement of the adductor longus and the sartorius. The feature quantity E4 is extracted from the section of the gait phase 11 to 16% of the gait waveform data related to the time-series data of the angular velocity in the horizontal plane (around the Z axis). The gait phase 11 to 16% is included in the mid-stance period T2. The feature quantity E4 mainly includes a feature related to the movement of the gluteus medius. The feature quantity E5 is extracted from the section of the gait phase 57 to 58% of the gait waveform data related to the time-series data of the angular velocity in the horizontal plane (around the Z axis). The gait phase 57 to 58% is included in the pre-swing period T4. The feature quantity E5 mainly includes a feature related to movement of the adductor longus and the sartorius. The feature quantity E6 is extracted from the section of the gait phase 100% of the gait waveform data related to the time-series data of the angle (posture angle) in the horizontal plane (around the Z axis). The gait phase 100% is relevant to the timing of heel strike that is switched from the terminal swing period T7 to the initial stance period T1. The feature quantity of the gait waveform data in the gait phase 100% is relevant to a foot angle in a state where the sole is grounded. The feature quantity E6 mainly includes a feature related to the movement of the gluteus medius. The feature quantity E7 is a distance (rotating amount) between the traveling axis and the foot at a timing when the central axis of the foot is farthest from the traveling axis in the swing phase. The feature quantity E7 is a rotating amount standardized by the height of the subject. The feature quantity E7 mainly includes a feature related to the movement of the abductor and adductor muscles.

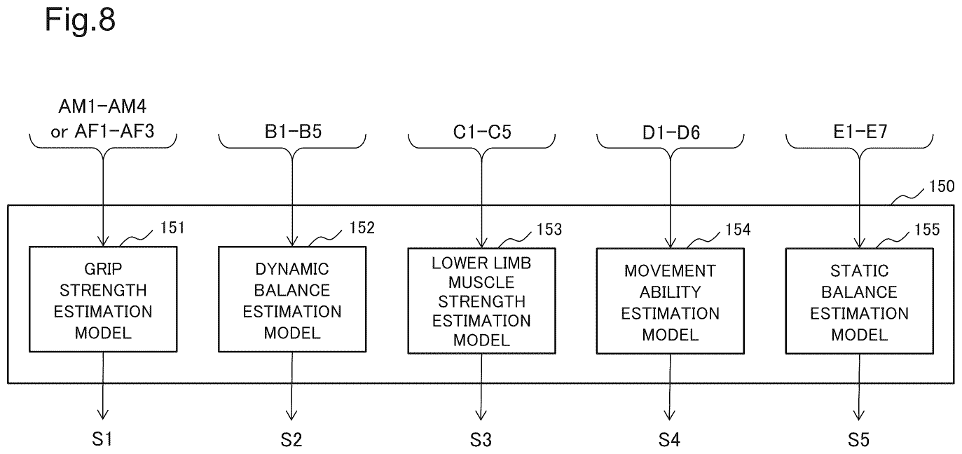

FIG. 8 is a conceptual diagram illustrating an example of a physical ability estimation model 150 for estimating physical ability. The feature quantity extracted from the gait waveform data is input to the physical ability estimation model 150 that estimates physical ability. In addition to the feature quantity data extracted from the gait waveform data, the attribute of the subject is input. In FIG. 8, the attributes input to the physical ability estimation model 150 are omitted. According to the input of the physical ability feature quantity extracted from the gait waveform data, the physical ability estimation model 150 outputs the physical ability score related to the physical ability. In the example of FIG. 8, the physical ability estimation model 150 includes a grip strength estimation model 151, a dynamic balance estimation model 152, a lower limb muscle strength estimation model 153, a movement ability estimation model 154, and a static balance estimation model 155. Each of the grip strength estimation model 151, the dynamic balance estimation model 152, the lower limb muscle strength estimation model 153, the movement ability estimation model 154, and the static balance estimation model 155 outputs a score for each estimation target of the model. The physical ability estimation model 150 may be configured by a single model instead of a model for each physical ability. The physical ability estimation model 150 may be a physical ability value such as a grip strength, an FR distance, a sit and stand time, a TUG required time, and a single-leg standing time, instead of the physical ability score.

The grip strength estimation model 151 outputs a grip strength score S1 related to the grip strength (total muscle strength of the whole body) according to the inputs of the feature quantities AM1 to AM4 or the feature quantities AF1 to AF3. For example, the grip strength estimation model 151 may be a model that outputs the grip strength according to the inputs of the feature quantities AM1 to AM4 or the feature quantities AF1 to AF3. For example, the grip strength estimation model 151 may be different models for men and women. The estimation result of the grip strength estimation model 151 is not limited as long as the estimation result related to the index of the grip strength is output according to the input of the physical ability feature quantity for estimating the total muscle strength. For example, the grip strength estimation model 151 may be a model that outputs the grip strength according to the inputs of the feature quantities AM1 to AM4 or the feature quantities AF1 to AF3. For example, the grip strength estimation model 151 may be a model that estimates the grip strength using attribute data such as age and height in addition to the feature quantities AM1 to AM4 or the feature quantities AF1 to AF3.

The dynamic balance estimation model 152 outputs a dynamic balance score S2 related to the dynamic balance according to the inputs of the feature quantities B1 to B5. The estimation result of the dynamic balance estimation model 152 is not limited as long as the estimation result related to the index of the dynamic balance is output according to the input of the physical ability feature quantity for estimating the dynamic balance. For example, the dynamic balance estimation model 152 may be a model that outputs the FR distance according to inputs of the feature quantities B1 to B5. For example, the dynamic balance estimation model 152 may be a model that estimates the dynamic balance using attribute data such as height in addition to the feature quantities B1 to B5.

The lower limb muscle strength estimation model 153 outputs a lower limb muscle strength score S3 related to lower limb muscle strength according to the inputs of the feature quantities C1 to C4. The estimation result of the lower limb muscle strength estimation model 153 is not limited as long as the estimation result related to the index of the lower limb muscle strength is output according to the input of the physical ability feature quantity for estimating the lower limb muscle strength. For example, the lower limb muscle strength estimation model 153 may be a model that outputs the lower limb muscle strength score S3 related to the lower limb muscle strength according to the inputs of the feature quantities C1 to C4. For example, the lower limb muscle strength estimation model 153 may be a model that estimates the dynamic balance using attribute data such as age in addition to the feature quantities C1 to C4.

The movement ability estimation model 154 outputs a movement ability score S4 related to the movement ability according to the inputs of the feature quantities D1 to D6. The estimation result of the movement ability estimation model 154 is not limited as long as the estimation result related to the index of the movement ability is output according to the input of the physical ability feature quantity for estimating the movement ability. For example, the movement ability estimation model 154 may be a model that outputs the TUG required time according to the inputs of the feature quantities D1 to D6. For example, the movement ability estimation model 154 may be a model that estimates the movement ability using attribute data such as age in addition to the feature quantities D1 to D6.

The static balance estimation model 155 outputs a static balance score S5 related to the static balance according to the inputs of the feature quantities E1 to E7. The estimation result of the static balance estimation model 155 is not limited as long as the estimation result related to the index of the static balance is output according to the input of the physical ability feature quantity for estimating the static balance. For example, the static balance estimation model 155 may be a model that outputs the single-leg standing time according to inputs of the feature quantities E1 to E7. For example, the static balance estimation model 155 may be a model that estimates the static balance using attribute data such as age and height in addition to the feature quantities E1 to E7.

The physical ability estimation model 150 may be stored in an external storage device constructed in a cloud, a server, or the like. In that case, the physical ability estimation unit 125 uses the physical ability estimation model 150 via an interface (not illustrated) connected to the storage device. The physical ability estimation model 150 is a machine learning model. For example, the physical ability estimation model 150 is a model in which a data set having attributes and gait indexes related to a plurality of subjects as explanatory variables and a score related to physical ability as an objective variable is learned as training data. The physical ability estimation model 150 may be a model in which a data set having attributes and gait waveform data related to a plurality of subjects as explanatory variables and a score related to the physical ability as an objective variable is learned as training data. For example, the physical ability estimation model 150 may be a model obtained by learning training data in which gait waveform data of acceleration in three axis directions, angular velocity around three axes, and angle (posture angle) around three axes are included in explanatory variables.

For example, the physical ability estimation model 150 may be generated by learning using a linear regression algorithm. For example, the physical ability estimation model 150 may be generated by learning using an algorithm of a support vector machine (SVM). For example, the physical ability estimation model 150 may be generated by learning using a Gaussian process regression (GPR) algorithm. For example, the physical ability estimation model 150 may be generated by learning using a random forest (RF) algorithm. For example, the physical ability estimation model 150 may be generated by unsupervised learning that classifies the subject from whom the physical ability feature quantity is generated according to the input of the physical ability feature quantity. The algorithm for learning the physical ability estimation model 150 is not particularly limited.

The disease risk estimation unit 126 (disease risk estimation) acquires an estimation result (physical ability score) of the physical ability estimated by the physical ability estimation unit 125. The disease risk estimation unit 126 acquires the gait index from the gait index calculation unit 123. Furthermore, the disease risk estimation unit 126 acquires the attribute of the subject from the storage unit 124. The disease risk estimation unit 126 estimates the disease risk for each disease using the physical ability score, the gait index, and the attribute. For example, the disease risk estimation unit 126 may be configured to estimate the disease risk for each disease using at least the gait index. The disease risk estimation unit 126 stores the estimated disease risk for each disease in the storage unit 124 in association with the subject. The estimated disease risk for each disease may be accumulated in a dedicated database (not illustrated) for generating a risk map.

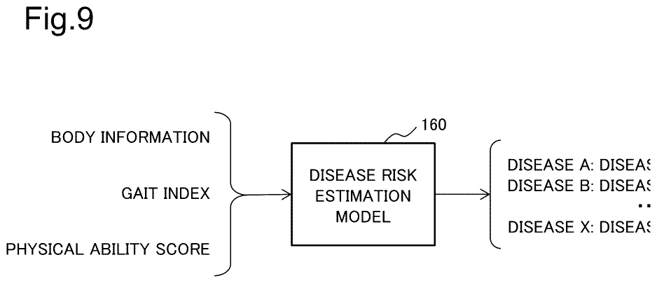

FIG. 9 is a conceptual diagram illustrating an example of disease risk estimation by the disease risk estimation unit 126. The disease risk estimation unit 126 inputs attribute data, a gait index, and a physical ability score used for estimating a disease risk related to a specific disease to a disease risk estimation model 160. Attribute data, a gait index, and a physical ability score used for estimating a disease risk related to a specific disease are input to the disease risk estimation model 160. According to the input of the attribute data, the gait index, and the physical ability score, the disease risk estimation model 160 outputs the disease risk score related to the specific disease. In the example of FIG. 9, the disease risk score is estimated for each of a plurality of diseases. The disease risk estimation model 160 may be configured by a model for each disease or may be configured by a single model. As the number of pieces of data used for estimation increases, the estimation accuracy of the disease risk score by the disease risk estimation model 160 improves.

For example, the disease risk estimation model 160 outputs a disease risk score related to a specific disease such as a lifestyle-related disease. For example, the disease risk estimation model 160 outputs disease risk scores related to specific diseases such as gout, diabetes, hypertension, nephrolithiasis, liver cirrhosis, arteriosclerosis, thromboembolism, dyslipidemia, hypercholesterolemia, and hyperlipidemia. For example, the disease risk estimation model 160 includes low back pain, sleep apnea syndrome, insomnia, depression, knee osteoarthritis, Parkinson's syndrome, and the like. The disease risk estimation model 160 may be configured to output a disease risk score related to a disease other than the above. For example, the disease risk estimation model 160 may be configured to estimate the disease risk score including the test item data of the medical examination.

The disease risk estimation model 160 may be stored in an external storage device constructed in a cloud, a server, or the like. In that case, the disease risk estimation unit 126 uses the disease risk estimation model 160 via an interface (not illustrated) connected to the storage device. The disease risk estimation model 160 is a machine learning model. For example, the disease risk estimation model 160 is a model obtained by learning, as training data, a data set having attributes, a gait index, and physical ability related to a plurality of subjects as explanatory variables and a disease risk score related to a specific disease as an objective variable. For example, the disease risk estimation model 160 may be a model learned using training data in which gait waveform data of acceleration in three axis directions, angular velocity around three axes, and angle (posture angle) around three axes are included in explanatory variables.

For example, the disease risk estimation model 160 is generated by learning using a linear regression algorithm. For example, the disease risk estimation model 160 is generated by learning using an algorithm of a support vector machine (SVM). For example, the disease risk estimation model 160 is generated by learning using a Gaussian process regression (GPR) algorithm. For example, the disease risk estimation model 160 is generated by learning using a random forest (RF) algorithm. For example, the disease risk estimation model 160 may be generated by unsupervised learning that classifies a subject from whom the feature quantity data is generated according to the feature quantity data. The algorithm for learning the disease risk estimation model 160 is not particularly limited.

For example, the disease risk estimation model 160 may be a machine learning model such as an incomplete heterogeneous variational autoencoder or a random forest. In the case of the incomplete heterogeneous variational autoencoder, the disease risk of the subject can be estimated even if there are some missing values in the attribute data, the gait index, the physical ability score, and the like.

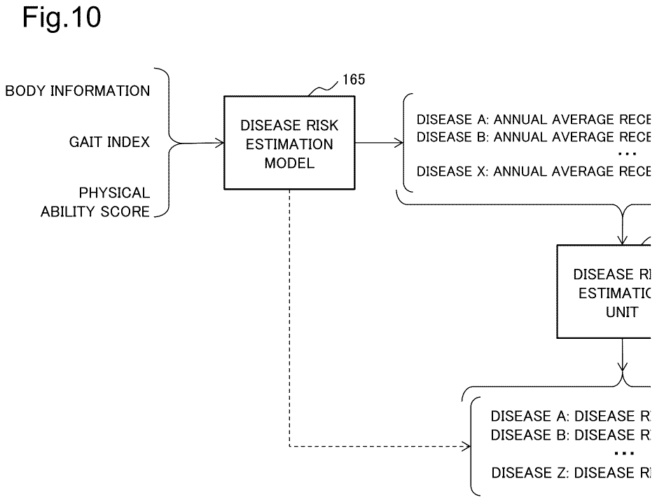

FIG. 10 is a conceptual diagram illustrating an example of a disease risk estimation model 165 that estimates the annual average receipt issuance number. The disease risk estimation unit 126 inputs the attribute data, the gait index, and the physical ability score to the disease risk estimation model 165. The attribute data, the gait index, and the physical ability score used for estimating a disease risk related to a specific disease are input to the disease risk estimation model 165. According to the input of the attribute data, the gait index, and the physical ability score, the disease risk estimation model 165 outputs the annual average receipt issuance number related to the specific disease. In the example of FIG. 10, the annual average receipt issuance number is estimated for each of a plurality of diseases. The disease risk estimation unit 126 calculates a disease risk score using the annual average receipt issuance number output from the disease risk estimation model 165. As the disease risk score, the annual average receipt issuance number may be used.

Here, an example in which the disease risk estimation unit 126 calculates the disease risk score using the annual average receipt issuance number will be described. Hereinafter, three calculation examples will be described. It is assumed that an annual average receipt issuance number μ₀ of a standard person is obtained in advance. The disease risk estimation model 165 outputs an annual average receipt issuance number u related to the specific disease according to the input of the attribute data, the gait index, and the physical ability score related to the estimation subject of the disease risk.

In the first method, the disease risk estimation unit 126 calculates, as the disease risk score, a ratio between the annual average receipt issuance number po of a standard person and the annual average receipt issuance number u estimated for the subject. The disease risk estimation unit 126 calculates a disease risk score RS₁ using the following Expression 1.

$$[ Math . 1 ]$$

$${RS}_{1} = \frac{{μ}_{0}}{μ} \qquad ( 1 )$$

In the second method, the disease risk estimation unit 126 calculates the disease risk score on the assumption that the annual average receipt issuance number for a specific disease follows the Poisson distribution. In the second method, the disease risk estimation unit 126 calculates a ratio between a probability mass function P₀(X=k) of the annual average receipt issuance number of a standard person and a probability mass function P(X=k) of the annual average receipt issuance number estimated for the subject as a disease risk score (k is a natural number). The disease risk estimation unit 126 calculates a disease risk score RS₂ using the following Expression 2.

$$[ Math . 2 ]$$

$${RS}_{2} = \frac{P ( X > 0 )}{{P}_{0} ( X > 0 )} = \frac{1 - exp ( - μ )}{1 - exp ( - {μ}_{0} )} \qquad ( 2 )$$

In the third method, the disease risk estimation unit 126 calculates an odds ratio of the annual average receipt issuance number for a specific disease. The disease risk estimation unit 126 calculates a disease risk score RS₃ using the following Expression 3.

$$[ Math . 3 ]$$

$${RS}_{3} = \frac{[ □ ( □ > 0 ) / \{ 1 - □ ( □ > 0 ) \} ]}{[ {□}_{0} ( □ > 0 ) / \{ 1 - {□}_{0} ( □ > 0 ) \} ]} = \frac{exp ( □ ) - 1}{exp ( {□}_{0} ) - 1} \qquad ( 3 )$$

The above three calculation examples are merely examples, and do not limit the method of calculating the disease risk score using the annual average receipt issuance number. The disease risk estimation unit 126 may be configured to calculate the disease risk score using an index other than the annual average receipt issuance number.

The map generation unit 127 acquires, from the storage unit 124, the disease risk of the disease for which the risk map is to be generated for the subject associated with the target district. For example, the position of the subject is specified by the address of the residence of the subject. For example, the position of the subject may be specified by position information acquired from a mobile terminal that is a transmission source of sensor data measured according to the movement of the foot of the subject. The map generation unit 127 acquires a map of the target district.

The map generation unit 127 sets a display condition of an image indicating the acquired distribution of the disease risk. The map generation unit 127 generates an image (heat map) in which a display state such as color coding or shading of the distribution of the disease risk is set in association with a position of a region, a house, or the like included in the target district. The map generation unit 127 sets the display state of the indicator according to the degree of the disease risk in association with the position of a region, a house, or the like included in the target district. For example, the map generation unit 127 sets indicators of display states such as hue, brightness, and saturation in accordance with the degree of the disease risk. The display state of the indicator according to the degree of the disease risk may be set according to a criterion other than hue, brightness, and saturation. For example, the map generation unit 127 may set a display state such as a shape, a size, and a color for each disease.

The map generation unit 127 sets a display state of an indicator indicating the degree of the disease risk in association with a region included in the target district. For example, the map generation unit 127 calculates a statistical value of the disease risk score for a plurality of subjects associated with a region included in the target district, and sets a display condition of an indicator relevant to the calculated statistical value for the region. For example, the statistic includes a total value, an average value, and the like of the disease risk scores. The regionalization of the target district is not particularly limited. For example, the map generation unit 127 divides the map of the target district at equal intervals. For example, the map generation unit 127 divides the map of the target district into grids. For example, map generation unit 127 sets a region in the map of the target district on a municipal basis. For example, the map generation unit 127 sets regions in units of residence indications (block, street number, etc.) obtained by dividing the map of the target district according to a block system or a road system.

The map generation unit 127 may set the display state of the indicator indicating the degree of the disease risk in association with the residence included in the target district. For example, the map generation unit 127 sets the display state for the subject associated with the residence included in the target district. For example, the map generation unit 127 calculates a statistic of the disease risk score for a plurality of subjects associated with a residence included in the target district, and sets a display condition of an indicator relevant to the calculated statistic for the residence. For example, the statistic includes a total value, an average value, and the like of the disease risk scores.

The map generation unit 127 may set a display state of an indicator indicating the degree of the disease risk in association with the position of the subject staying in the target district. For example, the map generation unit 127 sets the display state of the indicator indicating the degree of the disease risk in association with the position specified by the position information of the subject staying in the target district. For example, the map generation unit 127 updates the display state of the indicator indicating the degree of the disease risk in association with a change in the position specified by the position information of the subject. In that case, the risk map can be updated in accordance with the movement of the subject.

The map generation unit 127 superimposes the generated image (heat map) on the map of the target district according to the set display condition to generate the risk map. The map of the target district may be seen through the superimposed image (heat map). If it is known that the region or house included in the target district is associated with the subject, the indicator indicating the degree of the disease risk may deviate from the specified region or house and the position of the subject.

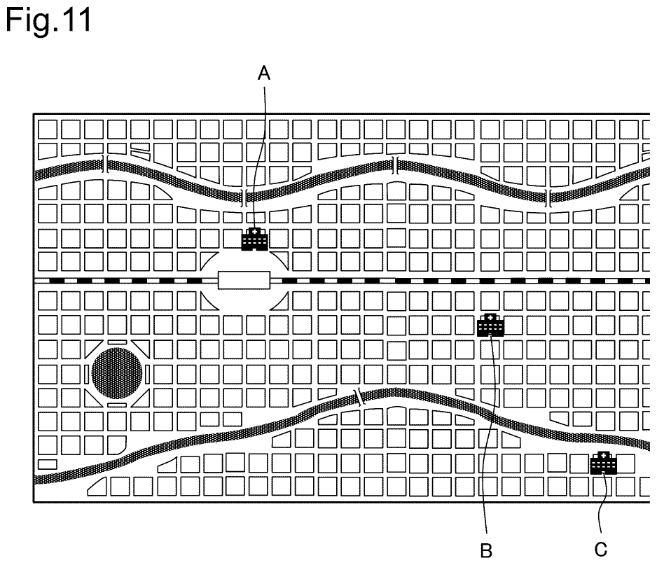

FIGS. 11 to 16 are conceptual diagrams for explaining the risk map generated by the map generation unit 127. FIG. 11 is a conceptual diagram showing an example (map M) of a map of a target district. The map M in FIG. 11 indicates positions of facilities (facility A, facility B, facility C, facility D) in the target district. As illustrated in FIG. 11, even in a facility in the target district, it may be difficult to access depending on a positional relationship between a residence of the subject and the facility. For example, a facility on the other side of a track or a river with respect to the position of the subject is close in distance, but is difficult to access.

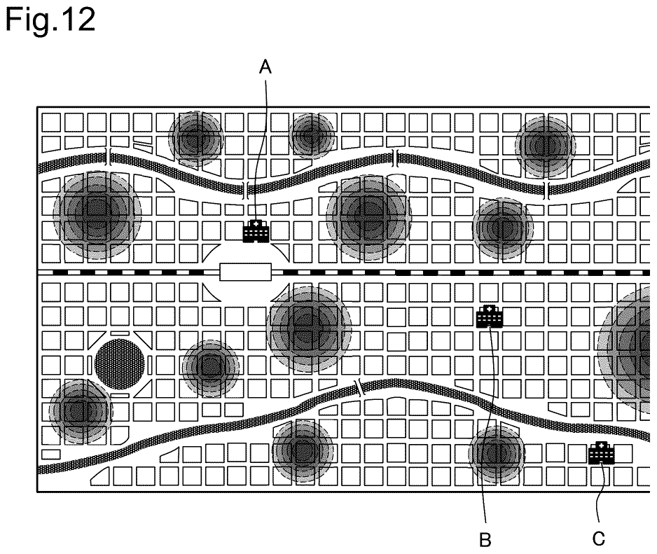

FIG. 12 is an example of a risk map (risk map RM1) in which an indicator indicating the degree of the disease risk of diabetes is displayed. In the risk map RM1, an indicator indicating the degree of the disease risk of diabetes is displayed in a circle (broken line). The indicator indicating the degree of the disease risk of diabetes may be expressed not by a closed figure such as a circle but by a continuous change in the entire risk map RM1. In the risk map RM1, the degree of disease risk of diabetes is indicated by shading. For example, the indicator is set to a darker display state as the degree of the disease risk of diabetes increases. For example, the indicator may be set to a larger display state as the degree of disease risk of diabetes is higher.

In the example of FIG. 12, a diabetes specialist is resident in the facility D. There is a region R₁ having a large region of the indicator on the other side of the railroad with respect to the facility D. Among the subjects who live in this region R₁, there is a high possibility that the number of people who receive treatment for diabetes increases in the future. Therefore, if a bridge is provided on the track between the facility D and the region R₁, it is easy to visit the facility D from the region R₁ even if the number of people who receive treatment for diabetes increases. For example, by referring to the risk map RM1, a clerical staff of the public office of the target district can obtain an opportunity to make a plan to provide a bridge on the track between the facility D and the region R₁. In the example of FIG. 12, if a diabetes specialist is resident in the facility B near the region R₁, the subject living in the region R₁ can easily visit the facility B for the treatment of diabetes. For example, the clerical staff of the public office of the target district can obtain an opportunity to make a plan to attract a diabetes specialist to the facility B with reference to the risk map RM1. In other words, the risk map RM1 can provide information for supporting decision-making of planning for the target district.

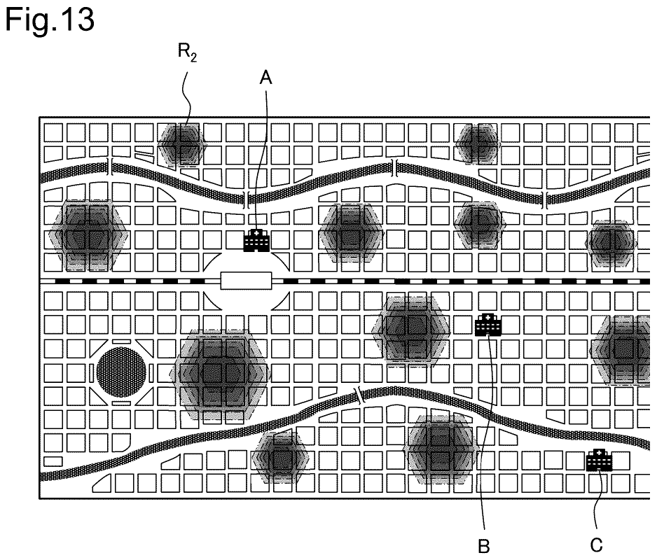

FIG. 13 is an example of a risk map (risk map RM2) in which an indicator indicating the degree of the disease risk of low back pain is displayed. In the risk map RM2, an indicator indicating the degree of the disease risk of low back pain is displayed in a hexagonal shape (one-dot chain line). The indicator indicating the degree of the disease risk of low back pain may be expressed by a continuous change in the entire risk map RM2 instead of a closed figure such as a hexagon. In the risk map RM2, the degree of the disease risk of low back pain is indicated by shading. For example, the indicator is set to a darker display state as the degree of the disease risk of low back pain increases. For example, the indicator may be set to a larger display state as the degree of disease risk of low back pain is higher.

In the example of FIG. 13, the facility C is a special hospital for low back pain. It is assumed that the facility C is away from the station and is currently away from the bus route. For example, transportation means such as a taxi is used to go from the region R₁ to the facility C. In order to go to the facility C also from other regions, transportation means such as a taxi is used. For example, by referring to the risk map RM2, a clerical staff of the public office of the target district can obtain an opportunity to make a plan to provide a bus route near the facility C. In the example of FIG. 13, if there is a bus going around the inside of the target region to go to the facility C, it is easy to visit the facility C. For example, by referring to the risk map RM2, a clerical staff of the public office of the target district can obtain an opportunity to make a plan for constructing a bus route that goes around the inside of the target region. In other words, the risk map RM2 can provide information for supporting decision-making of planning for the target district.

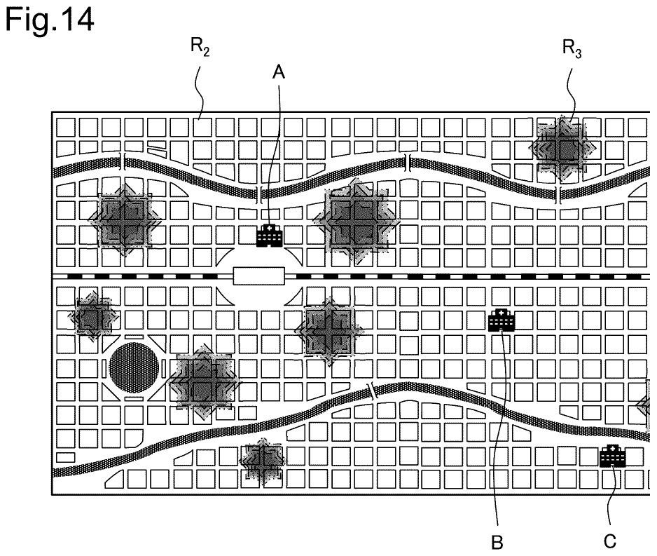

FIG. 14 is an example of a risk map (risk map RM3) in which an indicator indicating the degree of the disease risk of knee osteoarthritis is displayed. In the risk map RM3, an indicator indicating the degree of the disease risk of knee osteoarthritis is displayed as a star polygon (two-dot chain line). The indicator indicating the degree of the disease risk of knee osteoarthritis may be expressed by a continuous change in the entire risk map RM3 instead of a closed figure such as a star polygon. In the risk map RM3, the degree of the disease risk of knee osteoarthritis is indicated by shading. For example, the indicator is set to be in a darker display state as the degree of the disease risk of knee osteoarthritis increases. For example, the indicator may be set to a larger display state as the degree of the disease risk of knee osteoarthritis is higher. For example, as the degree of the disease risk of knee osteoarthritis is higher, the number of angles of the star polygon may be set to be larger in the display state.

In the example of FIG. 14, the facility A is a rehabilitation facility for knee osteoarthritis. Since the facility A is close to the station, it is easy for residents living in a region near the station to go to the facility A. However, transportation means such as a taxi are used to go from the region R₃ to the facility A. In order to go to the facility A also from other regions, transportation means such as a taxi is used. For example, by referring to the risk map RM3, a clerical staff of public office of the target district can obtain an opportunity to make a plan to attract a rehabilitation facility for knee osteoarthritis in the vicinity of a region where the indicator is large. In the example of FIG. 14, if there is a bus going around the inside of the target region and heading to the facility A, it is easy to visit the facility A. For example, by referring to the risk map RM3, a clerical staff of the public office of the target district can obtain an opportunity to make a plan for constructing a bus route that goes around the inside of the target region. In other words, the risk map RM3 can provide information for supporting decision-making of planning for the target district.

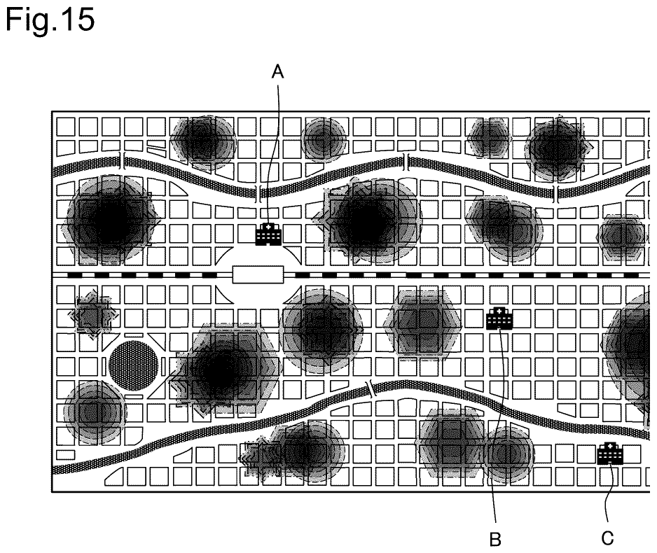

FIG. 15 is an example of a risk map (risk map RM4) in which indicators indicating the degrees of disease risks of a plurality of diseases are displayed. In the risk map RM4, an indicator indicating the degree of the disease risk of diabetes, low back pain, and knee osteoarthritis exemplified in FIGS. 12 to 14 is displayed. For example, if indicators indicating disease risks of diabetes, low back pain, and knee osteoarthritis are displayed in different shapes and colors, the distribution of these diseases can be distinguished. For example, when each of diabetes, low back pain, and knee osteoarthritis is not distinguished, the indicator indicating the disease risk may be displayed in the same shape or color. In the risk map RM3, the degree of the disease risk is indicated by shading. For example, the indicator is set to a darker display state as the degree of the disease risk increases. For example, the indicator may be set to a larger display state as the degree of disease risk is higher. For example, by referring to the risk map RM4, a clerical staff of the public office of the target district can obtain an opportunity to consider measures capable of comprehensively responding to a plurality of diseases. In other words, the risk map RM4 can provide information that supports decision-making of measures for the target district.

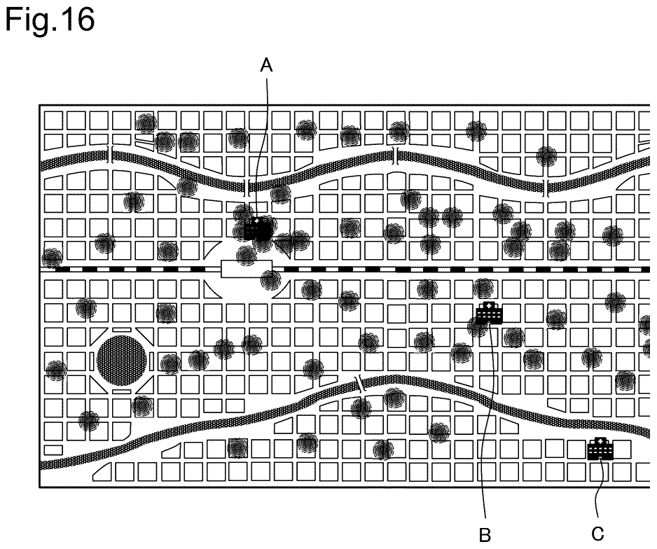

FIG. 16 is an example of a risk map (risk map RM5) in which an indicator indicating the degree of the disease risk of a disease is displayed in association with the position of the subject in the target district. The position of the subject is specified using position information of a mobile terminal (not illustrated) carried by the subject. An indicator indicating the degree of the disease risk of the subject is displayed at the position of the subject. For example, the indicator may be set to a darker display state as the degree of the disease risk increases. For example, the indicator may be set to a larger display state as the degree of disease risk is higher. For example, by referring to the risk map RM5, a clerical staff of the public office of the target district can grasp the disease risk of the subject in the target district in real time. For example, the clerical staff of the public office in the target district can get an opportunity to consider measures according to the moving situation of the subject having a disease risk. For example, the clerical staff of the public office in the target district can compare the tendency of the stay position in the daytime with the tendency of the stay position in the nighttime to obtain an opportunity to consider some measure. For example, by verifying the ratio of the at-home time and the going-out time, the clerical staff of the public office of the target district can obtain an opportunity to consider measures such as delivery of the delivery object and crime prevention. In other words, the risk map RM5 can provide information that supports decision-making of measures for the target district.

The output unit 129 (output means) outputs risk information including the risk map estimated by the map generation unit 127. For example, the output unit 129 outputs the risk information to a terminal device or a server managed by the public office of the target district. For example, the output unit 129 may display the risk information on a screen of a mobile terminal of the subject. For example, the output unit 129 may output the risk information to an external system or the like that uses the risk information. The use of the output risk information is not particularly limited. For example, the risk information is used for statistical analysis, disease prevention research, and the like.

For example, the information generation device 12 is connected to an external system or the like constructed in a cloud or a server via a mobile terminal (not illustrated) carried by the subject. The mobile terminal (not illustrated) is a portable communication device. For example, the mobile terminal is a portable communication device having a communication function, such as a smartphone, a smart watch, or a mobile phone. For example, the information generation device 12 is connected to a mobile terminal via wireless communication. For example, the information generation device 12 is connected to a mobile terminal via a wireless communication function (not illustrated) conforming to a standard such as Bluetooth (registered trademark) or WiFi (registered trademark). The communication function of the information generation device 12 may conform to a standard other than Bluetooth (registered trademark) or WiFi (registered trademark). For example, the information generation device 12 may be connected to a mobile terminal via a wire such as a cable. The disease risk information may be used by an application installed in the mobile terminal. In that case, the mobile terminal executes processing using the risk information by application software or the like installed in the mobile terminal.

### (Operation)

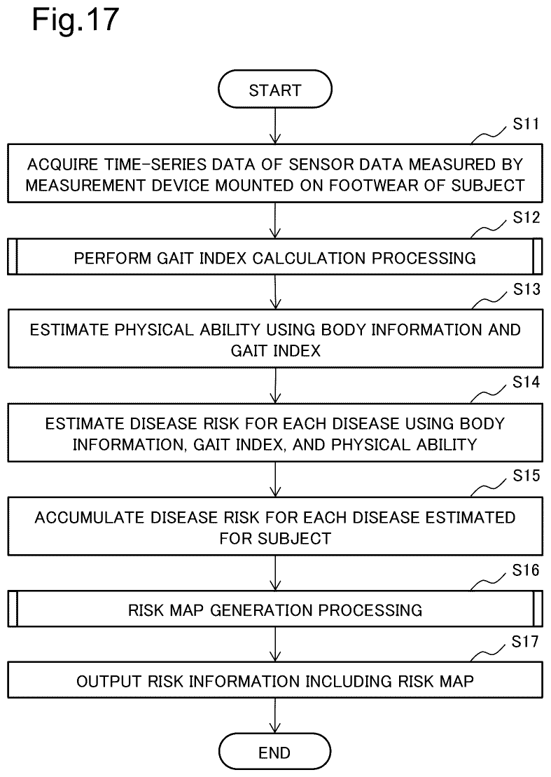

Next, an operation of the information provision system 1 will be described with reference to the drawings. Hereinafter, the operation of the information generation device 12 included in the information provision system 1 will be described. FIG. 17 is a flowchart for explaining an example of the operation of the information generation device 12. In the description of the processing along the flowchart of FIG. 17, the components of the information generation device 12 will be described as the operation subject. The operation subject of the processing along the flowchart of FIG. 17 may be the information generation device 12.

In FIG. 17, first, the acquisition unit 121 acquires time-series data of sensor data measured by the measurement device 10 mounted on the footwear (step S11). The sensor data includes acceleration in three axis directions and angular velocity around three axes.

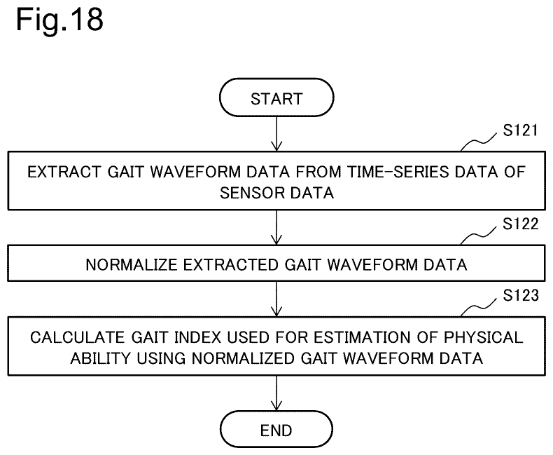

Next, the calculation unit 13 executes gait index calculation processing using the acquired sensor data (step S12). In the gait index calculation processing, the calculation unit 13 calculates a gait index used for estimating the physical ability. Details of the gait index calculation processing in step S12 will be described later (FIG. 18).

Next, the physical ability estimation unit 125 estimates the physical ability using the attribute data and the gait index (step S13). For example, the physical ability estimation unit 125 estimates physical ability scores such as grip strength (total muscle strength of the whole body), dynamic balance, lower limb muscle strength, movement ability, and static balance. In a case where the disease risk is estimated without using the physical ability, step S13 can be omitted.

Next, the disease risk estimation unit 126 estimates the disease risk for each disease using the attribute data, the gait index, and the physical ability (step S14). In a case where the disease risk is estimated without using the physical ability, the disease risk estimation unit 126 estimates the disease risk for each disease using the attribute data and the gait index. The disease risk estimation unit 126 estimates a disease risk score for each disease. For example, the disease risk estimation unit 126 estimates a disease risk score for each disease such as gout, diabetes, hypertension, nephrolithiasis, liver cirrhosis, arteriosclerosis, thromboembolism, dyslipidemia, hypercholesterolemia, or hyperlipidemia. For example, the disease risk estimation unit 126 estimates a disease risk score for each disease such as low back pain, sleep apnea syndrome, insomnia, depression, knee osteoarthritis, and Parkinson's syndrome.

Next, the storage unit 124 accumulates the estimated disease risk (step S15). The disease risk accumulated in the storage unit 124 is used for generating a risk map.

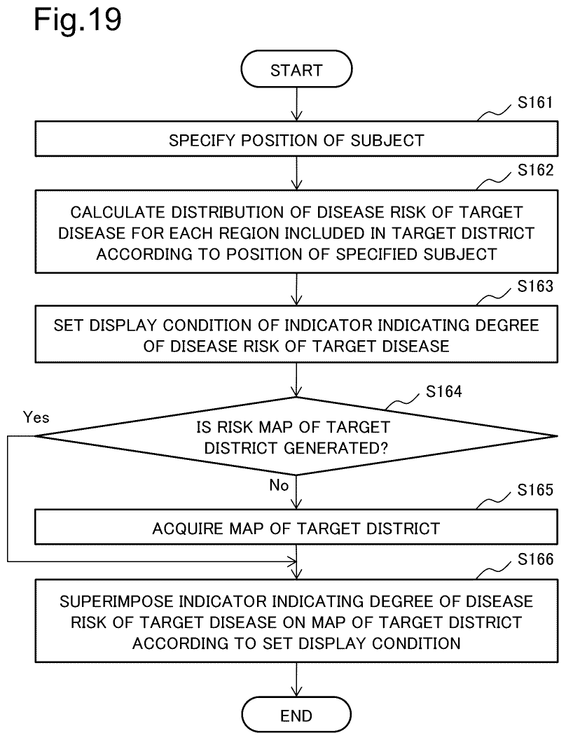

Next, the map generation unit 127 executes risk map generation processing using the disease risk accumulated in the storage unit 124 (step S16). The map generation unit 127 generates a risk map of the target district in the risk map generation processing. Details of the risk map generation processing in step S16 will be described later (FIG. 19).

Next, the output unit 129 outputs risk information including the generated risk map (step S17). For example, the output unit 129 outputs the risk information to a terminal device or a server managed by the public office of the target district. For example, the output unit 129 outputs the risk information to an external system or the like that uses the risk information. For example, the output unit 129 may display the risk information on a screen of a mobile terminal of the subject.

The attribute data of the subject may not necessarily be acquired. When the attribute data is not acquired from the subject, a model for estimating the disease risk without using the attribute data can be used. The consent to acquire the attribute data may be requested from the subject in advance. At that time, the subject may be notified of the merit obtained by acquiring the attribute data, and consent to the acquisition of the attribute data may be urged. Here, the merit is, for example, that a more accurate risk estimation result can be obtained.

### [Gait Index Calculation Processing]

Next, gait index calculation processing (step S12 in FIG. 17) by the calculation unit 13 of the information generation device 12 will be described with reference to the drawings. FIG. 18 is a flowchart for explaining an example of the operation of the calculation unit 13. In the description of the processing along the flowchart of FIG. 18, the components of the calculation unit 13 will be described as the operation subject. The operation subject of the processing along the flowchart of FIG. 18 may be the information generation device 12 or the calculation unit 13.

In FIG. 18, first, the waveform processing unit 122 extracts the gait waveform data from the time-series data of the sensor data (step S121). The gait waveform data is relevant to time-series data of sensor data for one gait cycle.

Next, the waveform processing unit 122 normalizes the extracted gait waveform data (step S122). The waveform processing unit 122 performs the first normalization on the gait waveform data in one gait cycle of 100%. The waveform processing unit 122 performs the second normalization on the gait waveform data so that the stance phase becomes 60% and the swing phase becomes 40%.

Next, the gait index calculation unit 123 calculates a gait index used for estimating the physical ability using the normalized gait waveform data (step S123). For example, the gait index calculation unit 123 calculates a gait index related to a distance, a height, an angle, a speed, a time, a frailty level, CPEI, and the like.

### [Risk Map Generation Processing]

Next, risk map generation processing (step S16 in FIG. 17) by the map generation unit 127 of the information generation device 12 will be described with reference to the drawings. FIG. 19 is a flowchart for explaining an example of the operation of the map generation unit 127. In the description of the processing along the flowchart of FIG. 19, the map generation unit 127 will be described as an operation subject. The operation subject of the processing along the flowchart of FIG. 19 may be the information generation device 12.

In FIG. 19, first, the map generation unit 127 specifies the position of the subject (step S161).

Next, the map generation unit 127 calculates the distribution of the disease risk of the target disease for each region included in the target district according to the position of the specified subject (step S162).

Next, the map generation unit 127 sets a display condition of an indicator indicating the degree of the disease risk of the target disease (step S163).

In a case where the risk map of the target district has not been generated (No in step S164), the map generation unit 127 acquires a map of the target district (step S165). In a case where the risk map of the target district has been generated (Yes in step S164), the process proceeds to step S166.

After step S165 or in the case of Yes in step S164, the map generation unit 127 superimposes an indicator indicating the degree of the disease risk of the target disease on the map of the target district in accordance with the set display condition (step S166). The map of the target district on which the indicator indicating the degree of the disease risk of the target disease is superimposed is the risk map.

### Application Example

Next, an application example according to the present example embodiment will be described with reference to the drawings. In the following application example, a relationship between a business operator that provides a service using the information provision system 1, a local government that uses the service, and a resident (subject) who lives in a management target district of the local government is illustrated.

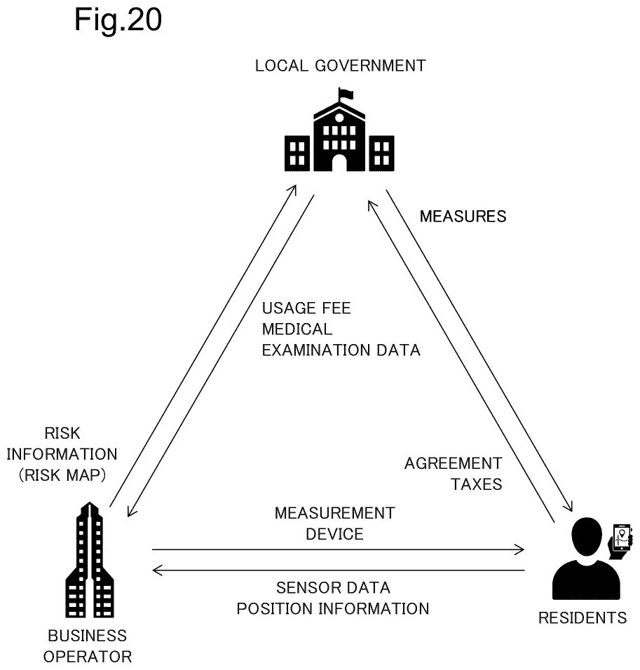

FIG. 20 is a correlation diagram illustrating a relationship among a business operator, a local government, and a resident (subject). The business operator provides services using the information provision system 1 to the local government. The business operator provides risk information including a risk map to a local government based on a contract concluded with the local government. The local government pays the service usage fee for the information provision system 1 to the business operator. In a case where the medical examination data of the resident is used to generate the risk map, the local government provides the medical examination data of the resident to the business operator. In the contract between the business operator and the local government, rules related to handling of personal information and appropriate data management are clarified. The business operator clearly explains that the risk information is reference information and does not guarantee medical accuracy or completeness.

The local government sufficiently explains the contents of the personal information protection policy and the data management to the residents, and then obtains consent from the residents. In a case where there is a change in the personal information protection policy or the content of data management, the local government shall explain to the residents and obtain their consent. For example, consent from residents is electronically performed. The local government takes measures for the district where the residents live according to the content of the risk information provided from the business operator.

The resident is a main entity that pays taxes to the local government. The resident receives a loan or a donation of a dedicated insole on which the measurement device 10 is mounted from a business operator contracted with the local government. The resident walks wearing shoes with a dedicated insole and carrying a mobile terminal capable of communicating with the measurement device 10. The mobile terminal uploads the sensor data measured by the measurement device 10 to the cloud server of the business operator.

A terminal device used in the local government downloads risk information of a target district from a cloud server of a business operator. The local government refers to the risk information. The local government refers to the risk map included in the risk information to consider measures for the residents. The local government periodically refers to the risk map of the target district and considers measures relevant to changes in the risk map. For example, the local government holds consultation meetings and events, and incorporates opinions and requests of residents with respect to measures relevant to changes in the risk map.

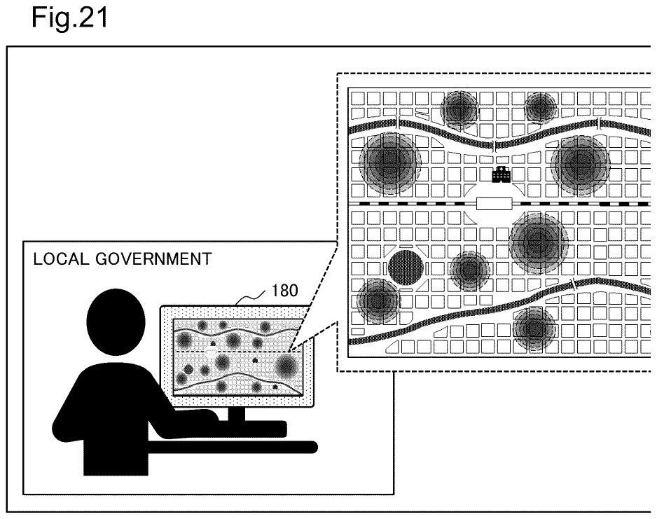

FIG. 21 illustrates an example in which a risk map is displayed on a screen of a terminal device 180 used in a local government. On the screen of the terminal device 180, risk information including a risk map generated for a target district managed by the local government is optimized and displayed for each local government. The clerical staff who has confirmed the risk information including the risk map displayed on the screen of the terminal device 180 can consider measures related to the target district.

As described above, the information provision system of the present example embodiment includes the measurement device and the information generation device. The measurement device is installed on footwear of at least one subject. The measurement device measures spatial acceleration and spatial angular velocity. The measurement device generates sensor data using the measured spatial acceleration and spatial angular velocity. The measurement device transmits the generated sensor data to the information generation device. The information generation device includes an acquisition unit, a risk estimation unit, a map generation unit, and an output unit. The acquisition unit acquires sensor data measured by a measurement device mounted on footwear of at least one subject. The risk estimation unit estimates a disease risk for each disease related to at least one subject using the acquired sensor data. The map generation unit generates a risk map in which a display relevant to the disease risk of the target disease related to at least one subject is superimposed on a map of the target district. The output unit outputs risk information including the generated risk map.

The information generation device according to the present example embodiment estimates the disease risk of the target disease using the sensor data measured by the measurement device mounted on the footwear of the subject. The information generation device according to the present example embodiment generates a risk map in which a display relevant to the estimated disease risk of the target disease is superimposed on a map of the target district. Therefore, according to the present example embodiment, it is possible to generate the risk map in which the position of the person at risk of acquiring the target disease is visualized.

In one aspect of the present example embodiment, the risk estimation unit includes a calculation unit and an estimation unit. The calculation unit calculates a gait index using the sensor data. The estimation unit inputs data including the gait index calculated using the sensor data to the disease risk estimation model, and estimates disease risk information relevant to the disease risk score output from the disease risk estimation model. The disease risk estimation model outputs a disease risk score indicating the degree of the disease risk for each disease according to the input of data including the gait index. According to the present aspect, the disease risk information relevant to the disease risk score can be estimated by inputting data including the gait index calculated using the sensor data to the disease risk estimation model.

In one aspect of the present example embodiment, the map generation unit specifies the position of at least one subject. The map generation unit calculates the distribution of the disease risk of the target disease for each region included in the target district according to the specified position of at least one subject. The map generation unit sets a display condition of an indicator indicating the degree of the disease risk of the target disease for each region included in the target district. The map generation unit superimposes an indicator indicating the degree of the disease risk of the target disease on the map of the target district according to the set display condition. According to the present aspect, it is possible to generate the risk map on which the indicator indicating the degree of the disease risk of the target disease is superimposed.

In one aspect of the present example embodiment, the map generation unit sets a display condition of an indicator indicating the degree of the disease risk of a plurality of target diseases for each region included in the target district. The map generation unit superimposes an indicator indicating the degree of the disease risk of the plurality of target diseases on the map of the target district according to the set display condition. According to the present aspect, it is possible to generate a risk map in which indicators related to a plurality of target diseases are displayed.

In one aspect of the present example embodiment, the map generation unit calculates a statistic of the disease risk score for at least one subject associated with the position inside the region included in the target district. The map generation unit sets an indicator display condition relevant to the calculated statistic of the disease risk score in the region. According to the present aspect, it is possible to generate the risk map on which the indicator relevant to the statistic of the disease risk score is displayed.

In one aspect of the present example embodiment, the map generation unit specifies the position of the subject based on the position of the residence of the subject. According to the present aspect, it is possible to generate the risk map in which the indicator indicating the degree of the disease risk of the target disease is displayed for each region relevant to the residence of the subject.

In one aspect of the present example embodiment, the map generation unit specifies the position of the subject based on position information of a mobile terminal carried by the subject. According to the present aspect, it is possible to generate the risk map in which the indicator indicating the degree of the disease risk of the target disease is displayed for each region relevant to the position of the mobile terminal carried by the subject.

In one aspect of the present example embodiment, the map generation unit specifies the position of the subject based on position information of a mobile terminal carried by the subject. The map generation unit updates the risk map according to a change in the position of the subject. According to the present aspect, the risk map on which the indicator indicating the degree of the disease risk of the target disease is displayed can be updated according to the change in the position of the mobile terminal carried by the subject.

In one aspect of the present example embodiment, the measure estimation model and the disease risk estimation model are models learned using a machine learning method. The disease risk estimation model includes an incomplete heterogeneous variational autoencoder. According to the present aspect, the disease risk of the subject can be estimated even if there is some loss in data such as the gait index.

In one aspect of the present example embodiment, the information generation device optimizes and displays risk information related to a target district for each local government on a screen of a terminal device used by the local government that manages the target district. According to the present aspect, the risk information including the risk map and the measure proposal of the target district can be optimized and provided for each local government that manages the target district.

### Second Example Embodiment

Next, an information generation device according to a second example embodiment will be described with reference to the drawings. The information generation device according to the present example embodiment generates a measure proposal relevant to the risk map. The information generation device of the present example embodiment outputs risk information including a measure proposal for the local government.

### (Configuration)

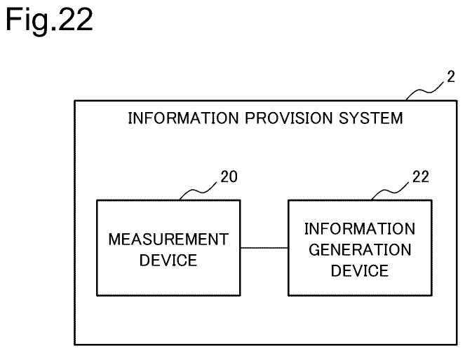

FIG. 22 is a block diagram illustrating an example of a configuration of an information provision system 2 in the present disclosure. The information provision system 2 includes a measurement device 20 and an information generation device 22. For example, the measurement device 20 is installed on the footwear of the subject who is an estimation target of a disease risk. The measurement device 20 has the same configuration as the measurement device 10 of the first example embodiment. Hereinafter, the description of the measurement device 20 will be omitted, and the information generation device 22 will be described. The main configuration of the information generation device 22 is similar to the configuration of the information generation device 12 of the first example embodiment, and thus the description thereof may be omitted.

### [Information Generation Device]

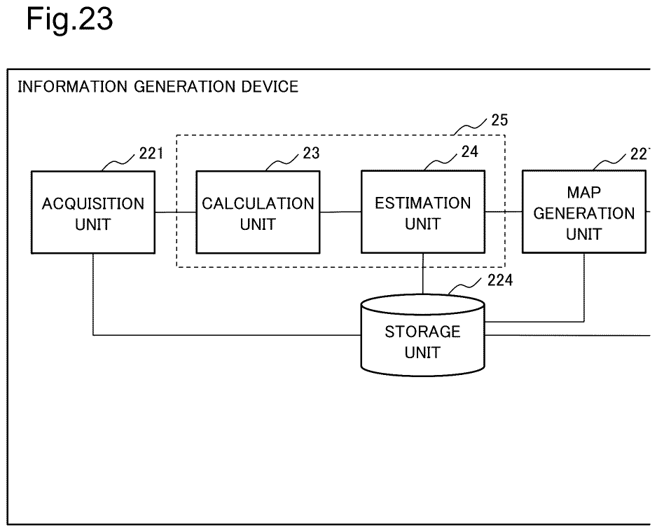

FIG. 23 is a block diagram illustrating an example of a configuration of the information generation device 22. The information generation device 22 includes an acquisition unit 221, a calculation unit 23, an estimation unit 24, a storage unit 224, a map generation unit 227, a measure proposal generation unit 228, and an output unit 229. The calculation unit 23 and the estimation unit 24 constitute a risk estimation unit 25.

The acquisition unit 221 (acquisition means) has the same configuration as the acquisition unit 121 of the first example embodiment. The acquisition unit 221 acquires sensor data from the measurement device 20 mounted on the footwear of the subject who uses the information provision system 2. The acquisition unit 221 receives sensor data from the measurement device 20 via wireless communication. The sensor data includes position information of a mobile terminal (not illustrated) of the subject who is a transmission source of the sensor data. For example, the acquisition unit 221 receives sensor data from the measurement device 20 via a wireless communication function (not illustrated) conforming to a standard such as Bluetooth (registered trademark) or WiFi (registered trademark). The communication function of the acquisition unit 221 may conform to a standard other than Bluetooth (registered trademark) or WiFi (registered trademark) as long as the communication function can communicate with the measurement device 20. The acquisition unit 221 may receive the sensor data from the measurement device 20 via a wire such as a cable. For example, the acquisition unit 221 may acquire a gait index or a feature quantity calculated by the measurement device 20.

The acquisition unit 221 acquires the attribute of the subject. The attribute data includes gender, date of birth, height, and weight. The date of birth is converted to age. The attribute data includes the address (position information) of the residence of the subject. The address (position information) of the residence of the subject is used to generate the risk map of the target district. Usually, the address (position information) of the residence of the subject is not used for estimating the physical ability or the disease risk. For example, the attribute data is input via an input device (not illustrated). For example, the attribute data is input via a mobile terminal used by the subject. For example, the attribute data may be stored in the storage unit 224 in advance. The attribute data may be updated at an arbitrary timing according to the input by the subject.

The calculation unit 23 (calculation means) has the same configuration as the calculation unit 13 of the first example embodiment. The calculation unit 23 has the functions of the waveform processing unit 122 and the gait index calculation unit 123 of the first example embodiment. The calculation unit 23 acquires sensor data from the acquisition unit 221. The calculation unit 23 extracts time-series data (gait waveform data) for one gait cycle from the time-series data of the acceleration in the three-axis direction and the angular velocity around the three axes included in the sensor data. The calculation unit 23 extracts the gait waveform data based on the timing of the gait event detected from the time-series data of the sensor data. For example, the calculation unit 23 extracts the gait waveform data with the timing of the heel strike as a start point and the timing of the next heel strike as an end point.

The calculation unit 23 normalizes the time of the extracted gait waveform data for one gait cycle to a gait cycle of 0 to 100% (percent) (first normalization). The calculation unit 23 normalizes the gait waveform data subjected to the first normalization for one gait cycle so that the stance phase becomes 60% and the swing phase becomes 40% (second normalization).

The calculation unit 23 extracts a feature quantity (physical ability feature quantity) used to estimate the physical ability from the gait waveform data. The calculation unit 23 extracts a physical ability feature quantity used to estimate at least one physical ability. For example, the calculation unit 23 extracts a physical ability feature quantity used for estimation of at least one of physical abilities such as grip strength (total muscle strength of the whole body), dynamic balance, lower limb muscle strength, movement ability, and static balance. For example, the calculation unit 23 extracts the physical ability feature quantity for each gait phase cluster according to a preset condition.

The calculation unit 23 calculates a gait index used for estimating the physical ability using the normalized gait waveform data. For example, the calculation unit 23 calculates a gait index related to a distance, a height, an angle, a speed, a time, a frailty level, a center of pressure exclusion index (CPEI), and the like.

The storage unit 224 (storage means) has the same configuration as the storage unit 124 of the first example embodiment. The storage unit 224 stores a physical ability estimation model for estimating the physical ability using the physical ability feature quantity extracted from the gait waveform data. For example, the physical ability estimation model outputs an index (physical ability score) related to the physical ability according to the input of the physical ability feature quantity extracted from the gait waveform data. The storage unit 224 stores a disease risk estimation model for estimating a disease risk using the attribute data, the gait index, and the physical ability score. For example, the disease risk estimation model outputs an index related to the disease risk (disease risk score) according to the input of the attribute data, the gait index, and the physical ability score. Usually, the address (position information) of the residence of the subject included in the attribute data is not used for estimating the physical ability or the disease risk. For example, the disease risk estimation model may be a model that outputs the disease risk score according to the input of the gait index and the attribute data without using the physical ability score. In that case, the physical ability estimation model may not be used.

The storage unit 224 also stores a measure estimation model that outputs measures for a target district according to an input of the risk map generated by the map generation unit 227. For example, the measure is information in which keywords related to the measure are associated with positions included in the target district. For example, the measure proposal is information in which keywords of measures related to facilities included in the target district are associated. For example, the measure estimation model may include a large-scale language model that outputs a sentence including a measure according to the input of the risk map.

The storage unit 224 stores a physical ability estimation model, a disease risk estimation model, and a measure estimation model learned for a plurality of subjects. For example, the physical ability estimation model, the disease risk estimation model, and the measure estimation model may be stored in the storage unit 224 at the time of shipment of a product from a factory. The physical ability estimation model, the disease risk estimation model, and the measure estimation model may be stored in the storage unit 224 at a timing such as at the time of calibration before the subject uses the information generation device 22. For example, a physical ability estimation model, a disease risk estimation model, and a measure estimation model stored in a storage device (not illustrated) such as an external server may be used. In that case, it is sufficient that the physical ability estimation model, the disease risk estimation model, and the measure estimation model can be accessed via an interface (not illustrated) connected to the storage device. The storage unit 224 stores the attribute of the subject. The attribute data includes gender, date of birth (age), height, and weight. The attribute data includes the address (position information) of the residence of the subject. Usually, the address (position information) of the residence of the subject is not used for estimating the physical ability or the disease risk. The attribute data may be updated at an arbitrary timing.

Furthermore, the storage unit 224 stores a map of a target district for which the risk map is generated. The map of the target district may be stored in advance in the storage unit 224. For example, the map of the target district may not be stored in the storage unit 224 and may be acquired from an external database by the acquisition unit 221.

The estimation unit 24 (estimation means) has the same configuration as the estimation unit 14 of the first example embodiment. The estimation unit 24 includes the functions of the physical ability estimation unit 125 and the disease risk estimation unit 126 according to the first example embodiment. The estimation unit 24 acquires the physical ability feature quantity extracted from the gait waveform data from the calculation unit 23. The estimation unit 24 acquires the attributes stored in the storage unit 224. The estimation unit 24 estimates the physical ability score using the physical ability feature quantity and the attribute. The estimation unit 24 inputs the physical ability feature quantity and the attribute of the subject to the physical ability estimation model stored in the storage unit 224. For example, the estimation unit 24 estimates a physical ability score related to the physical ability of at least one of grip strength (total muscle strength of the whole body), dynamic balance, lower limb muscle strength, movement ability, or static balance. The estimation unit 24 estimates the disease risk score for each disease using the physical ability score, the gait index, and the attribute. The estimation unit 24 inputs the physical ability score, the gait index, and the attribute to the disease risk model to estimate the disease risk score. The estimation unit 24 outputs the estimated disease risk score.

The map generation unit 227 has the same configuration as the map generation unit 127 of the first example embodiment. The map generation unit 227 acquires, from the storage unit 224, the disease risk of the disease for which the risk map is to be generated for the subject associated with the target district. The map generation unit 227 acquires a map of the target district.

The map generation unit 227 sets a display condition of an image indicating the acquired distribution of the disease risk. The map generation unit 227 generates an image (heat map) in which a display state such as color coding or shading of the distribution of the disease risk is set in association with a position of a region, a house, or the like included in the target district. The map generation unit 227 sets the display state of the indicator according to the degree of the disease risk in association with the position of a region, a house, or the like included in the target district.

The map generation unit 227 sets a display state of an indicator indicating the degree of the disease risk in association with a region included in the target district. The map generation unit 227 may set the display state of the indicator indicating the degree of the disease risk in association with the residence included in the target district. The map generation unit 227 may set a display state of an indicator indicating the degree of the disease risk in association with the position of the subject staying in the target district. The map generation unit 227 superimposes the generated image (heat map) on the map of the target district according to the set display condition to generate the risk map.

The measure proposal generation unit 228 acquires a risk map of the target district. The measure proposal generation unit 228 inputs the acquired risk map to the measure estimation model. The measure proposal generation unit 228 generates risk information including a measure proposal related to the measure using the measure output from the measure estimation model according to the input of the risk map.

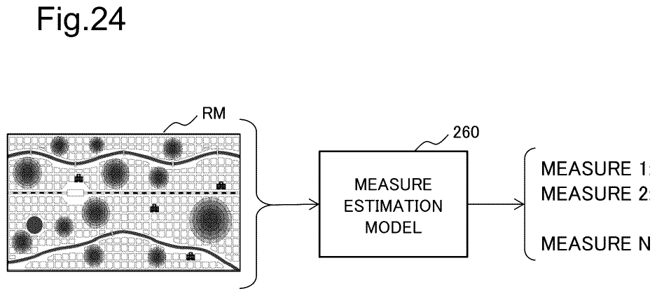

FIG. 24 is a conceptual diagram illustrating an estimation example of measures using a measure estimation model 260. The measure proposal generation unit 228 inputs the risk map of the target district to the measure estimation model 260. A risk map of the target district is input to the measure estimation model 260. According to the input of the risk map of the target district, the measure estimation model 260 outputs a measure of the target district. In the example of FIG. 24, a plurality of measures (measure 1, measure 2, . . . , measure N) are estimated for a target district (N is a natural number). In addition to the risk map of the target district, a map of the target district may be input to the measure estimation model 260. In that case, the measure estimation model 260 can estimate the measure after extracting the degree of the disease risk according to the indicator according to the difference between the risk map and the map of the target district.

For example, the measure proposal generation unit 228 generates risk information including a measure proposal related to a target district for a local government that manages the target district. For example, the measure proposal generation unit 228 generates a measure proposal by applying the measure to a preset document format. For example, the measure proposal generation unit 228 may generate a measure proposal using a large-scale language model. The local government can take an action relevant to the measure proposal in response to the acquisition of the risk information including the measure proposal. In other words, the measure proposal generation unit 228 generates risk information for supporting the decision-making of the local government.

The output unit 229 (output means) has the same configuration as the output unit 129 of the first example embodiment. The output unit 229 outputs risk information including the measure proposal generated by the measure proposal generation unit 228. The output unit 229 may also output risk information including the risk map. For example, the output unit 229 outputs risk information including a measure proposal to an external system or the like that uses the measure proposal. For example, the output unit 229 outputs risk information including a measure proposal to a terminal device (not illustrated) used in a local government. The use of the risk information including the output measure proposal is not particularly limited. For example, the risk information including a measure proposal is used for consideration of measures taken by the local government for the target district.

### (Operation)

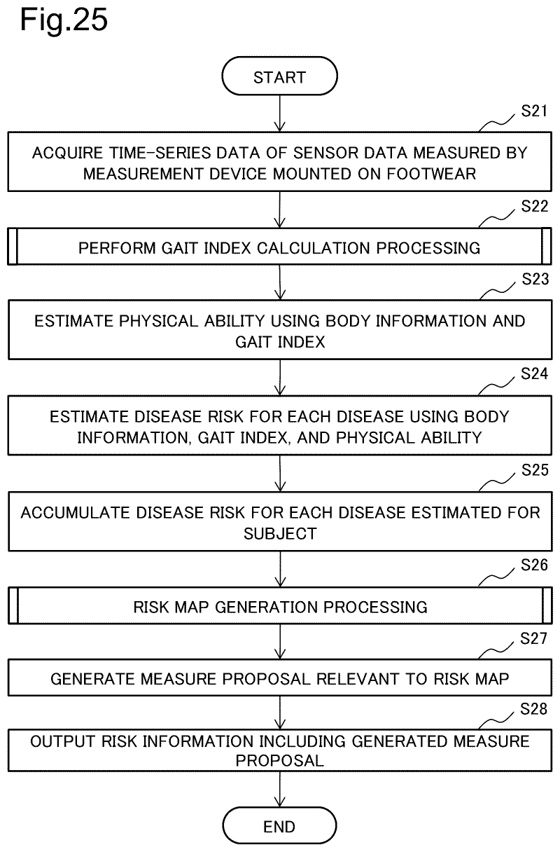

Next, an operation of the information provision system 2 will be described with reference to the drawings. Hereinafter, the operation of the information generation device 22 included in the information provision system 2 will be described. FIG. 25 is a flowchart for explaining an example of the operation of the information generation device 22. In the description of the processing along the flowchart of FIG. 25, the components of the information generation device 22 will be described as the operation subject. The operation subject of the processing along the flowchart of FIG. 25 may be the information generation device 22.

In FIG. 25, first, the acquisition unit 221 acquires time-series data of sensor data measured by the measurement device 20 mounted on the footwear (step S21). The sensor data includes acceleration in three axis directions and angular velocity around three axes.

Next, the calculation unit 23 executes gait index calculation processing using the acquired sensor data (step S22). In the gait index calculation processing, the calculation unit 23 calculates a gait index used for estimating the physical ability. The gait index calculation processing in step S22 is similar to the gait index calculation processing of the first example embodiment (FIG. 18).

Next, the estimation unit 24 estimates the physical ability using the attribute data and the gait index (step S23). For example, the estimation unit 24 estimates physical ability scores such as grip strength (total muscle strength of the whole body), dynamic balance, lower limb muscle strength, movement ability, and static balance. In a case where the disease risk is estimated without using the physical ability, step S23 can be omitted.

Next, the estimation unit 24 estimates the disease risk for each disease using the attribute data, the gait index, and the physical ability (step S24). In a case where the disease risk is estimated without using the physical ability, the estimation unit 24 estimates the disease risk for each disease using the attribute data and the gait index. The estimation unit 24 estimates a disease risk score for each disease. For example, the estimation unit 24 estimates a disease risk score for each disease such as gout, diabetes, hypertension, nephrolithiasis, liver cirrhosis, arteriosclerosis, thromboembolism, dyslipidemia, hypercholesterolemia, or hyperlipidemia. For example, the estimation unit 24 estimates a disease risk score for each disease such as low back pain, sleep apnea syndrome, insomnia, depression, knee osteoarthritis, and Parkinson's syndrome.

Next, the storage unit 224 accumulates the estimated disease risk (step S25). The disease risk accumulated in the storage unit 224 is used for generating a risk map.

Next, the map generation unit 227 executes risk map generation processing using the disease risk accumulated in the storage unit 224 (step S26). The map generation unit 227 generates a risk map of the target district in the risk map generation processing. The risk map generation processing of step S26 is similar to the risk map generation processing (FIG. 19) of the first example embodiment.

Next, the measure proposal generation unit 228 generates a measure proposal according to the risk map (step S27). The measure proposal generation unit 228 generates risk information including a measure proposal related to the measure using the measure output from the measure estimation model according to the input of the risk map.

Next, the output unit 229 outputs risk information including measure proposals (step S28). For example, the output unit 229 outputs the risk information to a terminal device or a server managed by the public office of the target district. For example, the output unit 229 outputs the risk information to an external system or the like that uses the risk information. For example, the output unit 229 may display the risk information on a screen of a mobile terminal of the subject.

### Application Example

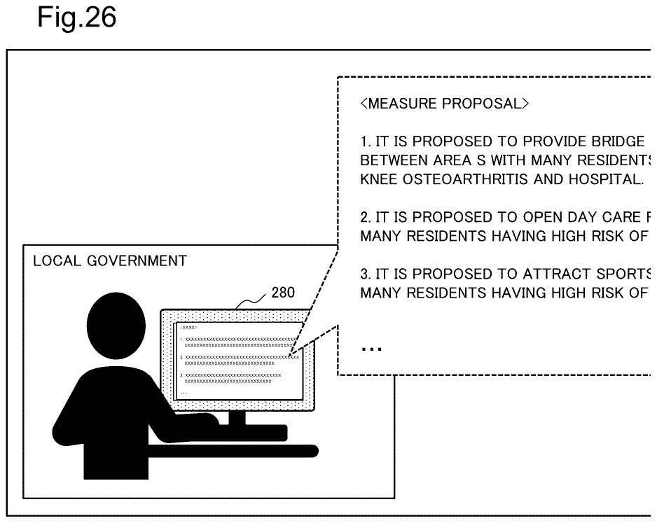

Next, an application example according to the present example embodiment will be described with reference to the drawings. FIG. 26 illustrates an example in which a measure proposal generated by the information generation device 22 is displayed on the screen of a terminal device 280 used in the local government. On the screen of the terminal device 280, risk information including proposal information generated for a target district managed by the local government is optimized and displayed for each local government. In the example of FIG. 26, a plurality of measure proposals are displayed on the screen. The first measure proposal is a proposal that “It is proposed to provide a bridge on a railway track between an area S with many residents having a high risk of knee osteoarthritis and a hospital”. The second measure proposal is a proposal that “It is proposed to open a day care facility in an area T with many residents having a high risk of frailty”. The third measure proposal is a proposal that “It is proposed to attract a sports gym to an area U with many residents having a high risk of diabetes”. The clerical staff who has confirmed the risk information including the measure proposal displayed on the screen of the terminal device 280 can consider measures related to the target district.

The measure proposal generated by the information generation device 22 is not limited to the above example as long as the measure proposal includes the measure related to the target district. For example, the measure proposal includes measures related to establishment and transfer of a medical facility such as a new clinic or pharmacy. For example, the measure proposal includes measures related to holding an event or a seminar related to healthcare leading to reduction of a health risk of a resident living in the target district. For example, the measure proposal includes a measure related to optimization of arrangement of the medical institution. For example, the measure proposal includes a city design measure related to the number of commercial facilities, geographical arrangement, installation of health equipment in a park, and the like. For example, the measure proposal includes a measure related to enhancement of public facilities such as a bus and a taxi so that access to a medical institution is improved. For example, the measure proposal includes measures related to opening and expansion of roads.

As described above, the information provision system of the present example embodiment includes the measurement device and the information generation device. The measurement device is installed on footwear of at least one subject. The measurement device measures spatial acceleration and spatial angular velocity. The measurement device generates sensor data using the measured spatial acceleration and spatial angular velocity. The measurement device transmits the generated sensor data to the information generation device. The information generation device includes an acquisition unit, a risk estimation unit, a map generation unit, a measure proposal generation unit, and an output unit. The acquisition unit acquires sensor data measured by a measurement device mounted on footwear of at least one subject. The risk estimation unit estimates a disease risk for each disease related to at least one subject using the acquired sensor data. The map generation unit generates a risk map in which a display relevant to the disease risk of the target disease related to at least one subject is superimposed on a map of the target district. The measure proposal generation unit generates a measure proposal relevant to the risk map of the target district using a measure estimation model that outputs a measure related to the target district according to the input of the risk map. The output unit outputs risk information including the generated measure proposal.

The information generation device according to the present example embodiment estimates the disease risk of the target disease using the sensor data measured by the measurement device mounted on the footwear of the subject. The information generation device according to the present example embodiment generates a risk map in which a display relevant to the estimated disease risk of the target disease is superimposed on a map of the target district. The information generation device according to the present example embodiment generates a measure proposal related to a target district using the generated risk map. Therefore, according to the present example embodiment, it is possible to generate a measure proposal reflecting the disease risk of at least one subject located in the target district.

### Third Example Embodiment

Next, an information generation device according to a third example embodiment will be described with reference to the drawings. The information generation device of the present example embodiment has a simplified configuration of the information generation device included in the information provision systems of the first and second example embodiments.

### (Configuration)

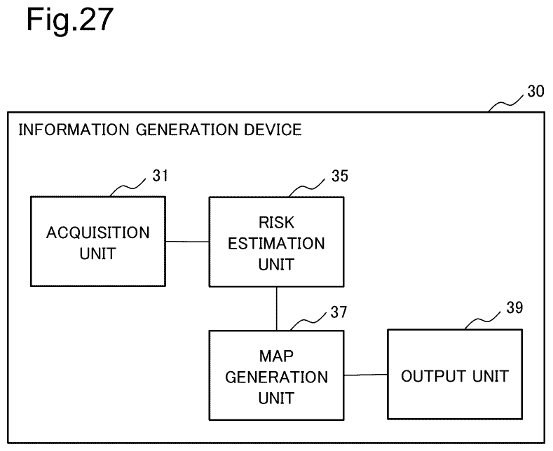

FIG. 27 is a block diagram illustrating an example of a configuration of an information generation device 30 in the present disclosure. The information generation device 30 includes an acquisition unit 31, a risk estimation unit 35, a map generation unit 37, and an output unit 39.

The acquisition unit 31 acquires sensor data measured by a measurement device mounted on footwear of at least one subject. The risk estimation unit 35 estimates a disease risk for each disease related to at least one subject using the acquired sensor data. The map generation unit 37 generates a risk map in which a display relevant to the disease risk of the target disease related to at least one subject is superimposed on a map of the target district. The output unit 39 outputs risk information including the generated risk map.

### (Operation)

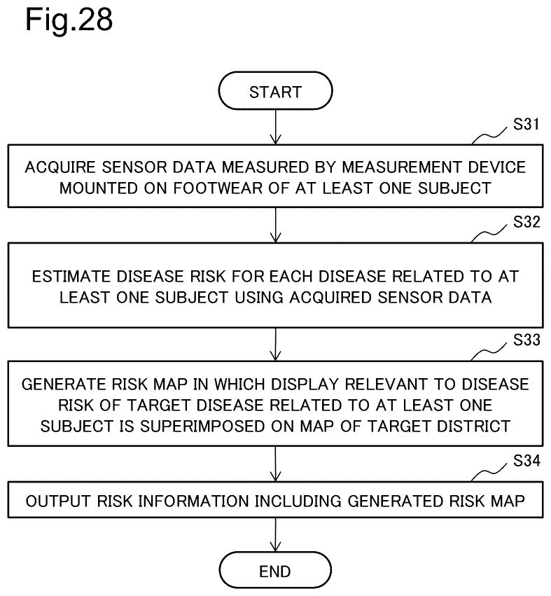

Next, an operation of the information generation device 30 will be described with reference to the drawings. FIG. 28 is a flowchart for explaining an example of the operation of the information generation device 30. In the description of the processing along the flowchart of FIG. 28, the components of the information generation device 22 will be described as the operation subject. The operation subject of the processing along the flowchart of FIG. 28 may be the information generation device 30.

In FIG. 28, first, the acquisition unit 31 acquires sensor data measured by a measurement device mounted on footwear of at least one subject (step S31).

Next, the risk estimation unit 35 estimates a disease risk for each disease related to at least one subject using the acquired sensor data (step S32).

Next, the map generation unit 37 generates a risk map in which a display relevant to the disease risk of the target disease related to at least one subject is superimposed on a map of the target district (step S33).

Next, the output unit 39 outputs risk information including the generated risk map (step S34).

As described above, the information generation device according to the present example embodiment estimates the disease risk of the target disease using the sensor data measured by the measurement device mounted on the footwear of the subject. The information generation device according to the present example embodiment generates a risk map in which a display relevant to the estimated disease risk of the target disease is superimposed on a map of the target district. Therefore, according to the present example embodiment, it is possible to generate the risk map in which the position of the person at risk of acquiring the target disease is visualized.

### (Hardware)

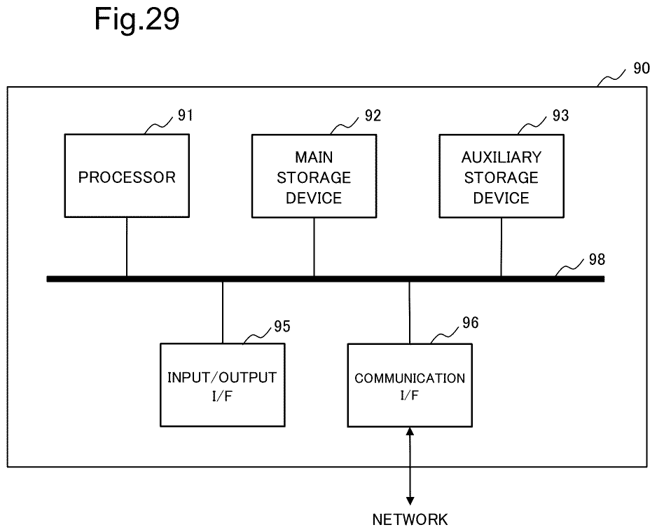

Next, a hardware configuration for executing control and processing according to each example embodiment of the present disclosure will be described with reference to the drawings. Here, an example of such a hardware configuration is an information processing device 90 (computer) in FIG. 29. The information processing device 90 of FIG. 29 is the configuration example for executing the control and processing in the present disclosure, and does not limit the scope of the present disclosure.

As illustrated in FIG. 29, the information processing device 90 includes a processor 91, a main storage device 92, an auxiliary storage device 93, an input/output interface 95, and a communication interface 96. In FIG. 29, the interface is abbreviated as an interface (I/F). The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program (instruction) stored in the auxiliary storage device 93 or the like in the main storage device 92. For example, the program is a software program for executing the control and processing in each embodiment. The processor 91 executes the program developed in the main storage device 92. The processor 91 executes the program to execute control and processing according to each example embodiment.

The main storage device 92 is a region in which a program is developed. A program stored in the auxiliary storage device 93 or the like is developed in the main storage device 92 by the processor 91. The main storage device 92 is achieved by, for example, a volatile memory such as a dynamic random access memory (DRAM). A nonvolatile memory such as a magneto resistive random access memory (MRAM) may be configured/added as the main storage device 92.

The auxiliary storage device 93 stores various types of data such as programs. The auxiliary storage device 93 is achieved by local disks such as hard disks or flash memories. Various types of data may be stored in the main storage device 92, and the auxiliary storage device 93 may be omitted.

The input/output interface 95 is an interface for connecting the information processing device 90 and a peripheral device in accordance with a standard or a specification. The communication interface 96 is an interface for connecting to an external system or device through a network such as the Internet or an intranet in accordance with a standard or a specification. The input/output interface 95 and the communication interface 96 may be shared as an interface connected to an external device.

Input devices such as a keyboard, a mouse, and a touch panel may be connected to the information processing device 90 as necessary. These input devices are used to input information and settings. In a case where the touch panel is used as the input device, a screen having a touch panel function serves as an interface. The processor 91 and the input device are connected via the input/output interface 95.

The information processing device 90 may be provided with a display device for displaying information. In a case where the display device is provided, the information processing device 90 includes a display control device (not illustrated) for controlling display of the display device. The information processing device 90 and the display device are connected via the input/output interface 95.

The information processing device 90 may be provided with a drive device. The drive device mediates reading of data and a program stored in a recording medium and writing of a processing result of the information processing device 90 to the recording medium between the processor 91 and the recording medium (program recording medium). The information processing device 90 and the drive device are connected via the input/output interface 95.

The above is an example of the hardware configuration for enabling the control and processing in the present disclosure. The hardware configuration of FIG. 29 is an example of the hardware configuration for executing the control and processing in the present disclosure, and does not limit the scope of the present disclosure. A program for causing a computer to execute control and processing according to each example embodiment is also included in the scope of the present disclosure.

A program recording medium in which the program according to each example embodiment is recorded is also included in the scope of the present disclosure. The recording medium can be achieved by, for example, an optical recording medium such as a compact disk (CD) or a digital versatile disk (DVD). The recording medium may be achieved by a semiconductor recording medium such as a universal serial bus (USB) memory or a secure digital (SD) card. The recording medium may be achieved by a magnetic recording medium such as a flexible disk, or other recording media. When a program executed by the processor is recorded in a recording medium, the recording medium is relevant to a program recording medium.

The components of the example embodiments may be arbitrarily combined. The components of each example embodiment may be achieved by software. The components of each example embodiment may be implemented by a circuit.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with other embodiments.

Some or all of the above example embodiments may also be described as the following Supplementary Notes, but are not limited to the following.

### Supplementary Note 1

An information generation device including:
an acquisition unit that acquires sensor data measured by a measurement device mounted on footwear of at least one subject;
a risk estimation unit that estimates a disease risk for each disease related to the at least one subject using the acquired sensor data;
a map generation unit that generates a risk map in which a display according to a disease risk of a target disease related to the at least one
subject is superimposed on a map of a target district; and an output unit that outputs risk information including the generated risk map.

### Supplementary Note 2

The information generation device according to Supplementary Note 1, in which
the risk estimation unit includes:
a calculation unit that calculates a gait index using the sensor data; and
an estimation unit that inputs data including the gait index calculated using the sensor data to a disease risk estimation model that outputs a disease risk score indicating a degree of a disease risk for each disease according to an input of the data including the gait index, and estimates disease risk information according to the disease risk score output from the disease risk estimation model.

### Supplementary Note 3

The information generation device according to Supplementary Note 2, in which
the map generation unit is configured to execute:
identifying a position of the at least one subject;
calculating a distribution of disease risks of the target disease for each region included in the target district according to positions of the at least one identified subject;
setting a display condition of an indicator indicating a degree of the disease risk of the target disease for each of the regions included in the target district; and
superimposing the indicator indicating the degree of the disease risk of the target disease on the map of the target district according to the set display condition.

### Supplementary Note 4

The information generation device according to Supplementary Note 3, in which
the map generation unit is configured to execute:
setting the display condition of the indicator indicating the degree of the disease risk of the plurality of target diseases for each of the regions included in the target district; and
superimposing the indicator indicating the degree of the disease risk of the plurality of target diseases on the map of the target district according to the set display condition.

### Supplementary Note 5

The information generation device according to Supplementary Note 3, in which
the map generation unit is configured to execute:
calculating a statistic of the disease risk score for the at least one subject associated with a position inside the region included in the target district; and
setting the display condition of the indicator according to the calculated statistic of the disease risk score to the region.

### Supplementary Note 6

The information generation device according to Supplementary Note 3, in which
the map generation unit is configured to execute:
specifying a position of the subject based on a position of a residence of the subject.

### Supplementary Note 7

The information generation device according to Supplementary Note 3, in which
the map generation unit is configured to execute:
specifying a position of the subject based on position information of a mobile terminal carried by the subject.

### Supplementary Note 8

The information generation device according to Supplementary Note 3, in which
the map generation unit is configured to execute:
specifying a position of the subject based on position information of a mobile terminal carried by the subject; and
updating the risk map according to a change in the position of the subject.

### Supplementary Note 9

The information generation device according to Supplementary Note 3, including:
a measure proposal generation unit that generates a measure proposal relevant to the risk map of the target district using a measure estimation model that outputs a measure related to the target district according to an input of the risk map, in which
the output unit is configured to execute:
outputting risk information including the generated measure proposal.

### Supplementary Note 10

The information generation device according to Supplementary Note 9, in which
the measure estimation model and the disease risk estimation model are models learned using a machine learning method, and
the disease risk estimation model includes an incomplete heterogeneous variational autoencoder.

### Supplementary Note 11

An information provision system including:
the information generation device according to any one of Supplementary Notes 1 to 10; and
a measurement device that is installed on footwear of the at least one subject, measures spatial acceleration and spatial angular velocity, generates the sensor data using the measured spatial acceleration and spatial angular velocity, and transmits the generated sensor data to the information generation device.

### Supplementary Note 12

The information provision system according to Supplementary Note 11, in which
the information generation device is configured to execute:
displaying the risk information optimized for the target district on a screen of a terminal device used by a local government that manages the target district.

### Supplementary Note 13

An information generation method for causing a computer to execute:
acquiring sensor data measured by a measurement device mounted on footwear of at least one subject;
estimating a disease risk for each disease related to the at least one subject using the acquired sensor data;
generating a risk map in which a display according to a disease risk of a target disease related to the at least one subject is superimposed on a map of a target district; and
outputting risk information including the generated risk map.

### Supplementary Note 14

The information generation method according to Supplementary Note 13, in which the computer executes:
calculating a gait index using the sensor data;
inputting data including the gait index calculated using the sensor data to a disease risk estimation model that outputs a disease risk score indicating a degree of a disease risk for each disease according to an input of the data including the gait index; and
estimating disease risk information according to the disease risk score output from the disease risk estimation model.

### Supplementary Note 15

The information generation method according to Supplementary Note 14, in which the computer executes:
identifying a position of the at least one subject;
calculating a distribution of disease risks of the target disease for each region included in the target district according to positions of the at least one identified subject;
setting a display condition of an indicator indicating a degree of the disease risk of the target disease for each of the regions included in the target district; and
superimposing the indicator indicating the degree of the disease risk of the target disease on the map of the target district according to the set display condition.

### Supplementary Note 16

The information generation method according to Supplementary Note 15, in which
the computer is configured to execute:
setting the display condition of the indicator indicating the degree of the disease risk of the plurality of target diseases for each of the regions included in the target district; and
superimposing the indicator indicating the degree of the disease risk of the plurality of target diseases on the map of the target district according to the set display condition.

### Supplementary Note 17

The information generation method according to Supplementary Note 15, in which
the computer is configured to execute:
calculating a statistic of a disease risk score indicating the disease risk for the at least one subject associated with a position inside the region included in the target district; and
setting the display condition of the indicator according to the calculated statistic of the disease risk score to the region.

### Supplementary Note 18

The information generation method according to Supplementary Note 15, in which
the computer is configured to execute:
specifying a position of the subject based on a position of a residence of the subject.

### Supplementary Note 19

The information generation method according to Supplementary Note 15, in which
the computer is configured to execute:
specifying a position of the subject based on position information of a mobile terminal carried by the subject.

### Supplementary Note 20

The information generation method according to Supplementary Note 15, in which
the computer is configured to execute:
specifying a position of the subject based on position information of a mobile terminal carried by the subject; and
updating the risk map according to a change in the position of the subject.

### Supplementary Note 21

The information generation method according to Supplementary Note 15, in which the computer executes:
generating a measure proposal relevant to the risk map of the target district using a measure estimation model that outputs a measure related to the target district according to an input of the risk map; and
outputting risk information including the generated measure proposal.

### Supplementary Note 22

The information generation method according to Supplementary Note 21, in which
the measure estimation model and the disease risk estimation model are models learned using a machine learning method, and
the disease risk estimation model includes an incomplete heterogeneous variational autoencoder.

### Supplementary Note 23

A non-transitory computer-readable recording medium having recorded therein a program for causing a computer to execute:
a process of acquiring sensor data measured by a measurement device mounted on footwear of at least one subject;
a process of estimating a disease risk for each disease related to the at least one subject using the acquired sensor data;
a process of generating a risk map in which a display according to a disease risk of a target disease related to the at least one subject is superimposed on a map of a target district; and
a process of outputting risk information including the generated risk map.

### Supplementary Note 24

The non-transitory computer-readable recording medium according to Supplementary Note 23 having recorded therein a program for causing a computer to execute:
a process of calculating a gait index using the sensor data;
a process of inputting data including the gait index calculated using the sensor data to a disease risk estimation model that outputs a disease risk score indicating a degree of a disease risk for each disease according to an input of the data including the gait index; and
a process of estimating disease risk information according to the disease risk score output from the disease risk estimation model.

### Supplementary Note 25

The non-transitory computer-readable recording medium according to Supplementary Note 24 having recorded therein a program for causing a computer to execute:
a process of identifying a position of the at least one subject;
a process of calculating a distribution of disease risks of the target disease for each region included in the target district according to positions of the at least one identified subject;
a process of setting a display condition of an indicator indicating a degree of the disease risk of the target disease for each of the regions included in the target district; and
a process of superimposing the indicator indicating the degree of the disease risk of the target disease on the map of the target district according to the set display condition.

### Supplementary Note 26

The non-transitory computer-readable recording medium according to Supplementary Note 25 having recorded therein a program for causing a computer to execute:
a process of setting a display condition of an indicator indicating a degree of the disease risk of a plurality of the target diseases for each of the regions included in the target district; and
a process of superimposing the indicator indicating the degree of the disease risk of a plurality of the target diseases on the map of the target district according to the set display condition.

### Supplementary Note 27

The non-transitory computer-readable recording medium according to Supplementary Note 25 having recorded therein a program for causing a computer to execute:
a process of calculating a statistic of the disease risk score indicating the disease risk for the at least one subject associated with a position inside the region included in the target district; and
a process of setting the display condition of the indicator according to the calculated statistic of the disease risk score to the region.

### Supplementary Note 28

The non-transitory computer-readable recording medium according to Supplementary Note 25 having recorded therein a program for causing a computer to execute a process of specifying a position of the subject based on a position of a residence of the subject.

### Supplementary Note 29

The non-transitory computer-readable recording medium according to Supplementary Note 15 having recorded therein a program for causing a computer to execute a process of specifying a position of the subject based on position information of a mobile terminal carried by the subject.

### Supplementary Note 30

The non-transitory computer-readable recording medium according to Supplementary Note 25 having recorded therein a program for causing a computer to execute:
a process of specifying a position of the subject based on position information of a mobile terminal carried by the subject; and
a process of updating the risk map according to a change in a position of the subject.

### Supplementary Note 31

The non-transitory computer-readable recording medium according to Supplementary Note 25 having recorded therein a program for causing a computer to execute:
a process of generating a measure proposal relevant to the risk map of the target district using a measure estimation model that outputs a measure related to the target district according to an input of the risk map; and
a process of outputting risk information including the generated measure proposal.

### Supplementary Note 32

The non-transitory computer-readable recording medium according to Supplementary Note 31, in which
the measure estimation model and the disease risk estimation model are models learned using a machine learning method, and
the disease risk estimation model includes an incomplete heterogeneous variational autoencoder.

### REFERENCE SIGNS LIST

1, 2 information provision system
10, 20 measurement device
12, 22 information generation device
13, 23 calculation unit
14, 24 estimation unit
15, 25 risk estimation unit
30 information generation device
31 acquisition unit
35 risk estimation unit
37 map generation unit
39 output unit
110 sensor
111 acceleration sensor
112 angular velocity sensor
113 control unit
115 communication unit
117 power supply
121, 221 acquisition unit
122 waveform processing unit
123 gait index calculation unit
124, 224 storage unit
125 physical ability estimation unit
126 disease risk estimation unit
127, 227 map generation unit
129, 229 output unit
228 measure proposal generation unit

What is claimed is:

1. An information generation device comprising:
a memory storing instructions; and
a processor connected to the memory and configured to execute the instructions to:
acquire sensor data measured by a measurement device mounted on footwear of at least one subject;
estimate a disease risk for each disease related to the at least one subject using the acquired sensor data;
generate a risk map in which a display according to a disease risk of a target disease related to the at least one subject is superimposed on a map of a target district; and
output risk information including the generated risk map.

2. The information generation device according to claim 1, wherein
the processor is configured to execute the instructions to:
calculate a gait index using the sensor data; and
input data including the gait index calculated using the sensor data to a disease risk estimation model that outputs a disease risk score indicating a degree of a disease risk for each disease according to an input of the data including the gait index, and estimates disease risk information according to the disease risk score output from the disease risk estimation model.

3. The information generation device according to claim 2, wherein
the processor execute is configured to execute the instructions to:
identify a position of the at least one subject;
calculate a distribution of disease risks of the target disease for each region included in the target district according to positions of the at least one identified subject;
set a display condition of an indicator indicating a degree of the disease risk of the target disease for each of the regions included in the target district; and
superimpose the indicator indicating the degree of the disease risk of the target disease on the map of the target district according to the set display condition.

4. The information generation device according to claim 3, wherein
the processor execute is configured to execute the instructions to:
set the display condition of the indicator indicating the degree of the disease risk of the plurality of target diseases for each of the regions included in the target district; and
superimpose the indicator indicating the degree of the disease risk of the plurality of target diseases on the map of the target district according to the set display condition.

5. The information generation device according to claim 3, wherein
the processor execute is configured to execute the instructions to:
calculate a statistic of the disease risk score for the at least one subject associated with a position inside the region included in the target district; and
set the display condition of the indicator according to the calculated statistic of the disease risk score to the region.

6. The information generation device according to claim 3, wherein
the processor execute is configured to execute the instructions to:
specify a position of the subject based on a position of a residence of the subject.

7. The information generation device according to claim 3, wherein
the processor execute is configured to execute the instructions to:
specify a position of the subject based on position information of a mobile terminal carried by the subject.

8. The information generation device according to claim 3, wherein
the processor execute is configured to execute the instructions to:
specify a position of the subject based on position information of a mobile terminal carried by the subject; and
update the risk map according to a change in the position of the subject.

9. The information generation device according to claim 3, wherein
the processor execute is configured to execute the instructions to:
generate a measure proposal relevant to the risk map of the target district using a measure estimation model that outputs a measure related to the target district according to an input of the risk map, and
output risk information including the generated measure proposal.

10. The information generation device according to claim 9, wherein
the measure estimation model and the disease risk estimation model are models learned using a machine learning method, and
the disease risk estimation model includes an incomplete heterogeneous variational autoencoder.

11. An information provision system comprising:
the information generation device according to claim 1; and
a measurement device that is installed on footwear of the at least one subject, wherein
the measurement device includes
a sensor that measures spatial acceleration and spatial angular velocity,
a controller that generates the sensor data using the measured spatial acceleration and spatial angular velocity, and
a transmitter that transmits the generated sensor data to the information generation device via wireless communication.

12. The information provision system according to claim 11, wherein
the processor of the information generation device is configured to execute the instructions to:
display the risk information optimized for the target district on a screen of a terminal device used by a local government that manages the target district.

13. An information generation method for causing a computer to execute:
acquiring sensor data measured by a measurement device mounted on footwear of at least one subject;
estimating a disease risk for each disease related to the at least one subject using the acquired sensor data;
generating a risk map in which a display according to a disease risk of a target disease related to the at least one subject is superimposed on a map of a target district; and
outputting risk information including the generated risk map.

14. The information generation method according to claim 13, wherein the computer executes:
calculating a gait index using the sensor data;
inputting data including the gait index calculated using the sensor data to a disease risk estimation model that outputs a disease risk score indicating a degree of a disease risk for each disease according to an input of the data including the gait index; and
estimating disease risk information according to the disease risk score output from the disease risk estimation model.

15. The information generation method according to claim 14, wherein the computer executes:
identifying a position of the at least one subject;
calculating a distribution of disease risks of the target disease for each region included in the target district according to positions of the at least one identified subject;
setting a display condition of an indicator indicating a degree of the disease risk of the target disease for each of the regions included in the target district; and
superimposing the indicator indicating the degree of the disease risk of the target disease on the map of the target district according to the set display condition.

16. The information generation method according to claim 15, wherein the computer executes:
generating a measure proposal relevant to the risk map of the target district using a measure estimation model that outputs a measure related to the target district according to an input of the risk map; and
outputting risk information including the generated measure proposal.

17. A non-transitory computer-readable recording medium having recorded therein a program for causing a computer to execute:
a process of acquiring sensor data measured by a measurement device mounted on footwear of at least one subject;
a process of estimating a disease risk for each disease related to the at least one subject using the acquired sensor data;
a process of generating a risk map in which a display according to a disease risk of a target disease related to the at least one subject is superimposed on a map of a target district; and
a process of outputting risk information including the generated risk map.

18. The non-transitory computer-readable recording medium according to claim 17 having recorded therein a program for causing a computer to execute:
a process of calculating a gait index using the sensor data;
a process of inputting data including the gait index calculated using the sensor data to a disease risk estimation model that outputs a disease risk score indicating a degree of a disease risk for each disease according to an input of the data including the gait index; and
a process of estimating disease risk information according to the disease risk score output from the disease risk estimation model.

19. The non-transitory computer-readable recording medium according to claim 18 having recorded therein a program for causing a computer to execute:
a process of identifying a position of the at least one subject;
a process of calculating a distribution of disease risks of the target disease for each region included in the target district according to positions of the at least one identified subject;
a process of setting a display condition of an indicator indicating a degree of the disease risk of the target disease for each of the regions included in the target district; and
a process of superimposing the indicator indicating the degree of the disease risk of the target disease on the map of the target district according to the set display condition.

20. The non-transitory computer-readable recording medium according to claim 19 having recorded therein a program for causing a computer to execute:
a process of generating a measure proposal relevant to the risk map of the target district using a measure estimation model that outputs a measure related to the target district according to an input of the risk map; and
a process of outputting risk information including the generated measure proposal.
